(19) United States
(12) Patent Application Publication
(10) Pub. No.: US 2026/0226189 A1
(43) Pub. Date: Aug. 6, 2026

(54) ANTI-CEA ANTIBODIES

(71) Applicant: PHILOGEN S.P.A., Siena (IT)

(72) Inventors: Louis PLÜSS, Otelfingen (CH); Roberto DE LUCA, Otelfingen (CH); Abdullah ELSAYED, Otelfingen (CH); Frederik PEISSERT, Otelfingen (CH); Dario NERI, Sovicille (SI) (IT)

(73) Assignee: PHILOGEN S.P.A., Siena (IT)

(21) Appl. No.: 19/153,172

(22) PCT Filed: Jan. 31, 2024
(86) PCT No.: PCT/EP2024/052429

§ 371 Date: Aug. 1, 2025

(30) Foreign Application Priority Data
Feb. 6, 2023 (EP) 23155224.1
May 12, 2023 (EP) 23173111.8

(51) Int. Cl. C07K 16/30; A61K 47/68; A61P 35/00; C07K 16/28

(52) U.S. Cl. CPC C07K 16/3007; A61K 47/6813; A61K 47/6853; A61K 47/6879; A61P 35/00; C07K 16/2809; C07K 16/2818; C07K 2317/31; C07K 2317/565; C07K 2317/622; C07K 2317/626

(57) ABSTRACT

Anti-CEA Antibodies The application relates to the diagnosis and treatment of diseases, including cancer. The invention provides, and involves the use of, antibody molecules that bind carcinoembryonic antigen (CEA) from humans. The antibody molecules may be conjugated to a pro-inflammatory agent, a biocidal molecule, a cytotoxic molecule, or a radioisotope. The antibody molecules may form part of a bispecific molecule which binds a T cell antigen, such as CD3.

This application claims priority from European Patent Application No. 23155224.1 filed 6 Feb. 2023, and European Patent Application No. 23173111.8 filed 12 May 2023, the contents and elements of both of which are herein incorporated by reference for all purposes.

### FIELD OF THE INVENTION

The present invention relates to the diagnosis and treatment of diseases, including cancer. The invention provides, and involves the use of, antibody molecules that bind human carcinoembryonic antigen (CEA). The antibody molecules may be conjugated to a pro-inflammatory agent, a biocidal molecule, a cytotoxic molecule, or a radioisotope. The antibody molecules may form part of a bispecific molecule which binds a T cell antigen, such as CD3.

### BACKGROUND

Carcinoembryonic antigen “CEA” is a GPI-anchored membrane protein, consisting of seven Ig-like domains. In healthy organs, CEA expression is restricted to the apical surface of mature enterocytes, where it participates in the formation of the glycocalyx, a protective layer in the intestinal lumen. In healthy individuals, CEA is inaccessible for antibodies in circulation.

In 98.8% of colorectal cancer tissues and various other malignancies, CEA becomes heavily overexpressed on the whole surface of tumor cells and consequently becomes exposed to the vasculature and lymphatic system. This selective accessibility makes CEA an ideal target for antibody-based therapeutics. This concept has been confirmed by numerous nuclear medicine studies, which have shown that CEA targeting products specifically accumulate in CEA expressing lesions, while leaving healthy organs unstained.

Several anti-CEA antibodies have been previously disclosed including “F33-104” (Sato et al., 1999), “MN3” and “MN15” (Blumental et al., 2005), “35A7” (Hammarstrom et al., 1989), “ZCE 025” (Habdel Nabi et al., 1990), “5F4” “34B 1” “26H7” (WO2013/082366) as well as others described in WO2010/125571, WO2012/082470 and WO2014/022332. However, as these antibodies are murine antibodies, they are liable to be immunogenic in humans and thus are not suitable for the treatment of human patients.

To decrease immunogenicity, some of the most promising murine anti-CEA antibodies have been converted into chimeric or humanized antibodies. For example, “T84.66” (Wagener et al., 1983) has been converted into a chimeric version “CT84.66” (Neumaier et al., 1990) and, later, into a humanized version “hT84.66” (WO2005/086875, Yazaki et al., 2004). However, despite the effort of humanizing this antibody, “hT84.66” still contains murine residues in the CDR2 of the heavy chain and has never been converted into a fully human antibody.

Murine anti-CEA antibody “MRG1” (WO2010/125571) has also been converted into a chimeric version “CM10” (WO2013/054331), but again has never been made into a fully human antibody.

Similarly, the chimeric anti-CEA antibody “MFE-23” (WO95/15431, Chester et al., 1994) has been converted into at least two humanized versions “hMFE-23” (Pedersen et al., 1994) and “sm3E” (US2005/0147614A1) but has never been converted into a fully human antibody.

The humanized anti-CEA antibody “MN-14” better known as “Labetuzumab” (WO03/033654, Stein and Goldenberg, 2004, Rijpkema et al., 2014, Hekman et al., 2017) the humanized chimeric antibody “X4” (Garambois et al., 2004), the chimeric antibody “CB/ior-Cea” (Perez et al., 2006), and the antibody fragment “scFv-96NRT”, which was isolated from an immune library of CDRs derived from cancer patients, immunized sheep or immunized mice (Qin et al., 2022), have also never been converted into fully human antibodies.

Fully human anti-CEA antibodies reported in the literature include “VG” (Garambois et al., 2004), “MA39” and “E8” (Pavoni et al., 2006). There remains a need in the art for additional fully human anti-CEA antibodies which are considered safe for the administration to human patients.

The present invention has been devised in light of the above considerations.

### SUMMARY OF THE INVENTION

The present inventors have isolated fully human antibody molecules which bind the N-terminal domain of human CEA with nanomolar affinity. The 7.7 nM binding affinity of the anti-CEA antibodies of the present invention compares favourably with the binding affinities of a number of other known fully human anti-CEA antibodies (VG, MA39 and E8; affinities set out below; see Example 1).

The antibodies of the present invention combine nanomolar affinity for the N-terminal domain of CEA with excellent biochemical and biophysical properties (Example 1), and target tumors with high efficiency (Examples 2 and 3), resulting in an excellent biodistribution profile (Example 3).

Furthermore, the antibodies of the present invention are fully human antibodies. Fully human antibodies are advantageous due to their lower potential for immunogenicity when administered to human patients.

The excellent biodistribution profile of the antibodies of the present invention and their fully human nature is expected to translate into a more favourable toxicity profile than that observed with other known anti-CEA antibodies, which is particularly advantageous in combination with their high affinity for CEA-expressing tumors.

In addition, other carcinoembryonic antigen-related cell adhesion molecules (CEACAMs), namely CEACAM1 and CEACAM6, have highly conserved N-terminal domains relative to CEA. The specificity of the antibodies of the present invention to CEA (CEACAM5) was confirmed by flow cytometry using CHO cells transiently expressing CEA (CEACAM5), CEACAM 1, or CEACAM6, whereby no binding was detected to CEACAM1- and CEACAM6-expressing cells (Example 8 and FIG. 13).

In a first aspect, the present invention thus relates to human antibody molecules that bind the N-terminal domain of carcinoembryonic antigen (CEA). “CEA” as referred to herein refers to CEACAM5. The sequence of the N-terminal domain of the human carcinoembryonic antigen (CEACAM5) is shown in SEQ ID NO: 1. The present invention thus relates to human antibody molecules that bind the N-terminal domain of CEACAM5. The human antibody molecules of the invention do not bind CEACAM1 and CEACAM6.

The antibody molecule of the invention preferably comprises the HCDR1, HCDR2, and HCDR3 sequences of the “PEA2” antibody set forth in SEQ ID NOs 2, 3 and 4, respectively, and/or the LCDR1, LCDR2 and LCDR3 sequences of the PEA2 antibody set forth in SEQ ID NOs 5, 6 and 7, respectively. The PEA2 antibody, which comprises these 6 CDR sequences, has been shown to be capable of binding the N-terminal domain of CEA with nanomolar affinity (Example 1). In contrast, the known human anti-CEA antibody “VG” has an affinity of 0.19±0.06×10⁻⁸ M⁻¹ in VG-IgG2κ format and 1.30±0.06×10⁻⁸ M⁻¹ in VG-IgM format, while the affinity of anti-CEA antibodies “MA39” and “E8” was measured to be 1.71×10⁻⁷ and 1.39×10⁻⁸, respectively.

In a preferred embodiment, the antibody molecule of the invention comprises the VH domain or VL domain sequence, but preferably the VH domain and VL domain sequence, of the PEA2 antibody molecule set forth in SEQ ID NOs 8 and 9, respectively. The VH and VL domains of the antibody molecule may be linked by a linker, such as the linker set forth in SEQ ID NO: 11.

The antibody molecule of the invention may be in any suitable format. Many antibody molecule formats are known in the art and include both complete antibody molecule molecules, such as IgG, as well as antibody fragments, such as a single chain Fv (scFv), diabodies, or single-chain diabodies. The term “antibody molecule” as used herein encompasses both complete antibody molecule molecules and fragments of antibody molecules, in particular antigen-binding fragments. In a preferred embodiment, the antibody molecule consists of or comprises an scFv, a small immunoprotein (SIP), a diabody, a single-chain diabody, or a (complete) IgG molecule, such as an IgG1 or IgG4 molecule. The sequence of the PEA2 antibody in single chain Fv (scFv) format is shown in SEQ ID NO: 10. The sequence of the PEA2 antibody in diabody (Db) format is shown in SEQ ID NO: 12. The sequence of the PEA2 antibody in single chain diabody (scDb) format is shown in SEQ ID NO: 13. The sequence of the PEA2 antibody in small immunoprotein (SIP) format is shown in SEQ ID NO: 41. The sequence of the PEA2 light chain in IgG format is shown in SEQ ID NO: 17, while the sequences of the PEA2 heavy chain in IgG1 format and IgG4 format are shown in SEQ ID NO: 16 and SEQ ID NO: 40 respectively.

In healthy organs, CEA expression is restricted to the apical surface of mature enterocytes, where it participates in the formation of the glycocalyx, a protective layer in the intestinal lumen. In healthy patients, CEA is inaccessible for antibodies in circulation. In 98.8% of colorectal cancer tissues and various other malignancies, CEA becomes heavily overexpressed on the whole surface of tumor cells and consequently becomes exposed to the vasculature and lymphatic system. This selective accessibility makes CEA an ideal target for antibody-based therapeutics.

In addition, CEA has been shown to be useful as a marker for cancers, particularly colorectal cancers, being localised at sites of disease with high specificity. The antibody of the invention may thus be employed in the imaging, detection and diagnosis of diseases and disorders characterised, or associated with, the expression of CEA, including cancers. In this context, the antibody molecule may be used as is and later detected using e.g. a secondary antibody molecule or may be conjugated to a detectable label.

An antibody molecule of the present invention may be used as is, i.e. in unconjugated form, or may be conjugated to a molecule to provide a conjugate. The choice of molecule conjugated to the antibody molecule will depend on the intended application of the conjugate. For example, where the conjugate is intended for the treatment of a disease or disorder, the conjugate may comprise an antibody molecule of the invention and a bioactive agent. The bioactive agent may be a pro-inflammatory agent. In a preferred embodiment, the bioactive agent is a pro-inflammatory agent, in particular, a cytokine. The conjugates of the present invention retain the binding affinity of PEA2 for CEA and the biological activity of the active moiety conjugated to the antibody (Examples 4-6).

In some embodiments, the conjugate of the present invention comprises an antibody molecule of the present invention conjugated to a cytokine selected from the group consisting of Interleukin-12 (IL12), Interleukin-2 (IL2), or Tumor Necrosis Factor alpha (TNFα), or a variant thereof. A cytokine may be conjugated to the N-terminus or C-terminus of the antibody molecule or both.

The sequence of a conjugate comprising the PEA2 antibody in single-chain diabody format conjugated at its N-terminus to IL12 is shown in SEQ ID NO: 26. The sequence of a conjugate comprising the PEA2 antibody in diabody format conjugated at its C-terminus to IL2 is shown in SEQ ID NO: 21. The sequence of a conjugate comprising the PEA2 antibody in scFv format conjugated at its C-terminus to TNFα is shown in SEQ ID NO: 28. Where a cytokine is conjugated to both the N-terminus and the C-terminus of the antibody molecule, the cytokines may be the same or different but preferably are different. The sequence of a conjugate comprising the PEA2 antibody in scFv format conjugated at its C-terminus to TNFα, wherein the scFv is further conjugated at its N-terminus to IL2, is shown in SEQ ID NO: 30.

Where the conjugate is intended for use in imaging, detecting, or diagnosing a disease or disorder, the conjugate may comprise an antibody molecule of the invention and a detectable label or marker molecule, such as a radioisotope, e.g., a non-therapeutic radioisotope. Depending on the molecule conjugated to the antibody molecule, the conjugate may be or may comprise a single-chain protein. Where the conjugate is a single-chain protein, the entire protein can be expressed as a single polypeptide or fusion protein. In this case, the molecule may be conjugated to the antibody molecule by means of a peptide linker. Fusion proteins have the advantage of being easier to produce and purify since they consist of a single species. This facilitates production of clinical-grade material. Alternatively, the molecule may be conjugated to the antibody molecule by means of a cleavable linker.

The antibody molecule of the present invention, may be a bispecific antibody molecule comprising a second antigen-binding site for a second target antigen. The second target antigen is preferably not CEA.

Examples of suitable bispecific antibody molecules for use in the context of the present invention can be found in Kontermann 2012 (page 186 FIG. 2) the content of which is incorporated herein by reference.

In one preferred embodiment, the bispecific antibody molecule is a bispecific T cell engager molecule comprising the PEA2 antibody in scFv format and an anti-CD3 antibody in scFv format. The sequence of such a bispecific antibody molecule is set forth in SEQ ID NO: 33.

In another preferred embodiment, the bispecific antibody molecule is an scDb-scFv and comprises the PEA2 antibody in scDb format and an anti-CD3 antibody in scFv format. The sequence of such a bispecific antibody molecule is set forth in SEQ ID NO: 34.

In a further preferred embodiment, the bispecific antibody molecule is an IgG4-scFv molecule comprising the heavy chain amino acid sequences set forth in SEQ ID NOs: 48 and 49, and the light chain amino acid sequence set forth in SEQ ID NO: 17.

In yet another preferred embodiment, the bispecific antibody molecule is an IgG4-(scFv)₂ molecule comprising the heavy chain amino acid sequence set forth in SEQ ID NO: 50, and the light chain amino acid sequence set forth in SEQ ID NO: 17.

In another preferred embodiment, the bispecific antibody molecule is a scDb-scFv with Fc knob-in-hole (KIH) molecule comprising the PEA2 antibody in scDb format and an anti-CD3 antibody in scFv format. The two heavy chain sequences of such a bispecific antibody molecule are set forth in SEQ ID NOs: 54 and 55, respectively.

In another preferred embodiment, the bispecific antibody molecule is a scFv-IgG KIH molecule comprising the PEA2 antibody in IgG4 KIH format and an anti-CD3 antibody in scFv format. The two heavy chain sequences of such a bispecific antibody molecule are set forth in SEQ ID NOs: 56, 57 and the light chain sequence is set forth in SEQ ID NO: 58.

In one preferred embodiment, the bispecific antibody molecule is an IgG-(scFv)₂ molecule comprising the PEA2 antibody in scFv format and an anti-CD28 in IgG4 format. The heavy chain sequence of such a bispecific antibody molecule is set forth in SEQ ID NO: 59 and the light chain amino acid sequence is set forth in SEQ ID NO: 60.

The invention also provides isolated nucleic acids encoding the antibody molecules and conjugates of the invention. The skilled person would have no difficulty in preparing such nucleic acids using methods well-known in the art. An isolated nucleic acid may be used to express the antibody molecule or conjugate of the invention, for example by expression in a bacterial, yeast, insect or mammalian host cell. A preferred host cell is E. coli. The nucleic acid will generally be provided in the form of a recombinant expression vector for expression. Host cells in vitro comprising such nucleic acids and expression vectors are part of the present invention, as is their use for expressing the antibody molecules and conjugates of the invention, which may subsequently be purified from cell culture and optionally formulated into a pharmaceutical composition.

An antibody molecule or conjugate of the invention may be provided for example in a pharmaceutical composition, and may be employed for medical use as described herein, either alone or in combination with one or more further therapeutic agents. For example, an antibody molecule or conjugate of the invention may be employed for a medical use as described herein in combination with a second therapeutic agent. Exemplary further therapeutic agents that may be combined with, or administered in association with, an antibody molecule or conjugate of the present invention include, e.g., chemotherapy (e.g., anti-cancer chemotherapy, for example, paclitaxel, docetaxel, vincristine, cisplatin, carboplatin or oxaliplatin), radiation therapy, a checkpoint inhibitor, e.g., a checkpoint inhibitor that targets PD-1 (e.g., an anti-PD-1 antibody such as pembrolizumab, nivolumab, or cemiplimab (see U.S. Pat. No. 9,987,500)), CTLA-4, LAG3, or TIM3, or a costimulatory agonist antibody that targets e.g. GITR, OX40, or 4-1 BB, or an immunoconjugate such as an immunocytokine, or a bispecific antibody.

A bispecific antibody molecule which binds CEA and T cell antigen may be administered in combination with a bispecific antibody molecule of the invention which binds CEA and CD3. The T cell antigen in this context is preferably not CD3. In a preferred embodiment, the T cell antigen is CD28. Thus, a bispecific antibody molecule which binds CEA and CD28 may be administered in combination with a bispecific antibody molecule of the invention which binds CEA and CD3. The anti-CEA antigen binding site of the bispecific anti-CEA anti-CD28 antibody molecule may bind the same epitope as the PEA2 antibody of the invention, or a different epitope, but preferably binds a different epitope on CEA than the PEA2 antibody of the invention.

Where the anti-CEA anti-CD28 antibody molecule binds the same epitope as the PEA2 antibody of the invention, the anti-CEA anti-CD28 antibody molecule may comprise the HCDR1, HCDR2, and HCDR3 and LCDR1, LCDR2 and LCDR3 sequences, and/or VH and VL sequences of the PEA2 antibody described herein.

Where the anti-CEA anti-CD28 antibody molecule binds a different epitope on CEA than the PEA2 antibody of the invention, the antibody molecule may comprise the HCDR1, HCDR2, and HCDR3 and LCDR1, LCDR2 and LCDR3 sequences, and/or VH and VL sequences of the anti-CEA antibody Sm3E shown herein, e.g. in the antibody molecule comprising SEQ ID NOs 61 and 62.

An example of an anti-CD28 antibody (AE2P) which may be incorporated into an anti-CEA anti-CD28 antibody molecule is described in PCT/EP2023/070320.

In one preferred embodiment, a bispecific antibody molecule of the invention binding CEA and CD3 in a scDb-scFv with Fc KIH format is administered in combination with an IgG-(scFv)₂ molecule comprising an anti-CD28 antibody in IgG4 format and anti-CEA antibody of the invention (PEA2) in scFv format. In a preferred example, a bispecific antibody molecule comprising, or consisting of, the sequences set forth in SEQ ID NOs: 54 and 55 is administered in combination with a bispecific antibody molecule comprising, or consisting of, the sequences set forth in SEQ ID NOs: 59 and 60.

In another preferred embodiment, a bispecific antibody molecule of the invention binding CEA and CD3 in a scDb-scFv with Fc KIH format is administered in combination with an IgG-(scFv)₂ molecule comprising an anti-CD28 antibody in IgG4 format and an anti-CEA antibody other than the antibody of the invention in scFv format (e.g. Sm3E). In a preferred example, a bispecific antibody molecule comprising, or consisting of, the sequences set forth in SEQ ID NOs: 54 and 55 is administered in combination with a bispecific antibody molecule comprising, or consisting of, the sequences set forth in SEQ ID NOs: 61 and 62.

In another preferred embodiment, a bispecific antibody molecule of the invention binding CEA and CD3 in a scFv-IgG KIH format is administered in combination with an IgG-(scFv)₂ molecule comprising an anti-CD28 antibody in IgG4 format and anti-CEA antibody of the invention (PEA2) in scFv format. In a preferred example, a bispecific antibody molecule comprising, or consisting of, the sequences set forth in in SEQ ID NOS: 56, 57 and 58 is administered in combination with a bispecific antibody molecule comprising, or consisting of, the sequences set forth in SEQ ID NOs: 59 and 60.

In another preferred embodiment, a bispecific antibody molecule of the invention binding CEA and CD3 in a scFv-IgG KIH format is administered in combination with an IgG-(scFv)₂ molecule comprising an anti-CD28 antibody in IgG4 format and an anti-CEA antibody other than the antibody of the invention (e.g. Sm3E) in scFv format. In a preferred example, a bispecific antibody molecule comprising, or consisting of, the sequences set forth in SEQ ID NOs: 56, 57 and 58 is administered in combination with a bispecific antibody molecule comprising, or consisting of, the sequences set forth in SEQ ID NOs: 61 and 62.

Alternatively, the antibody molecule or conjugate of the invention may be provided in a diagnostic composition and may be employed for diagnostic use as described herein.

The present invention also relates to an antibody molecule or conjugate of the invention for use in a method for treatment of the human or animal body by therapy. For example, an antibody molecule or conjugate of the invention may be for use in a method of treating cancer.

The invention also relates to a method of treating cancer in a patient, the method comprising administering a therapeutically effective amount of an antibody molecule or conjugate of the invention to the patient. The use of an antibody molecule or conjugate of the invention for the manufacture of a medicament for the treatment of cancer in a patient, is also contemplated.

The present invention further relates to an antibody molecule of the invention for use in a method of delivering a molecule to sites of cancer in a patient. The invention also relates to a method of delivering a molecule to sites of cancer in a patient comprising administering to the patient an antibody molecule of the invention, wherein the antibody molecule is conjugated to the molecule.

In a preferred embodiment, the antibody molecule or conjugate of the invention is for use in a method of treating cancer. Most preferably, the antibody molecule or conjugate of the invention is for use in a method of treating cancer, wherein the method further comprises administering an anti-PD-1 antibody to the patient.

A further aspect of the invention relates to an antibody molecule or conjugate of the invention for use in a method of imaging, detecting, or diagnosing cancer in a patient. The invention further relates to a method of imaging, detecting, or diagnosing cancer in a patient, optionally comprising administering an antibody molecule or conjugate of the invention to the patient. The method may be an in vitro or an in vivo method. Also encompassed within the scope of the invention is the use of an antibody molecule or conjugate of the invention for the manufacture of a diagnostic product for imaging, detecting, or diagnosing cancer.

In a preferred embodiment, the antibody molecule or conjugate of the invention is for use in a method of imaging, detecting, or diagnosing cancer.

A patient, as referred to herein, is preferably a human patient.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the characterisation of the anti-CEA antibodies G9, F7, and PEA2. Size exclusion chromatography and SDS-PAGE characterization under both reducing (R) and non-reducing (NR) conditions is shown for the antibodies G9, F7 and PEA2 in scFv format (FIG. 1A), diabody format (FIG. 1B) and IgG format (FIG. 1C). These results confirm the purity and correct molecular weight of the G9, F7, and PEA2 antibodies in the each of the three different formats.

FIG. 2 shows the affinity of the anti-CEA antibodies G9, F7 and PEA2 for the N-terminal domain of CEA. FIG. 2A shows the results of BIAcore analysis using monomeric scFv on a chip coated with human CEA antigen at four different concentrations of the G9, F7 and PEA2 scFvs: 1000 nM, 500 nM, 250 nM, and 125 nM. FIG. 2B shows the results of BIAcore analysis using G9, F7 and PEA2 antibodies in diabody format on a chip coated with human CEA antigen at 1000 nM. FIG. 2C shows the results of BIAcore analysis using G9, F7 and PEA2 antibodies in IgG format on a chip coated with human CEA antigen at 1000 nM. These results confirm the high affinity of PEA2 for the N-terminal domain of CEA.

FIG. 3 shows the results of immunofluorescence analysis evaluating the binding of the anti-CEA antibody PEA2 to CEA antigen. FIG. 3A shows the results of immunofluorescence staining of cryosections of LS174T tumor tissue slides using fluorescently-labelled anti-CEA antibody PEA2 IgG1-FITC and the anti-hen egg lysozyme antibody “KSF” (KSF IgG1-FITC; used as negative control). Anti-CEA antibody PEA2 IgG1-FITC was capable of recognizing the human CEA on frozen tissue slides. FIG. 3B shows the results of immunofluorescence staining of a human tissue microarray containing human cancer sections and corresponding healthy controls using anti-CEA antibody PEA2 IgG1-FITC. KSF IgG1-FITC was used as a negative control. Positive staining was obtained with PEA2 on CEA-positive colon, lung, and pancreatic tumors, showing PEA2 IgG1-FITC is capable of recognizing the human CEA on the human cancer sections.

FIG. 4 shows the results of flow cytometry analysis evaluating the binding of the anti-CEA antibodies G9, F7, and PEA2, each in IgG format, to CEA-expressing CT26 cells and to CEA-negative CT26 wild-type cells. The KSF antibody in IgG1 format was used as negative control. G9, F7, and PEA2 all showed binding to the CEA positive cell line. No binding was observed on the CEA-negative wild-type cells.

FIG. 5 shows the results of an ex vivo biodistribution analysis of fluorescently-labelled anti-CEA antibody PEA2 IgG1-FITC in LS174T human colon adenocarcinoma bearing mice. KSF IgG1-FITC was used as a negative control. Organs were harvested 24 hours after intravenous injection and distribution of the antibody was analyzed by immunofluorescence. Staining shows selective accumulation of PEA2 in tumor tissue with no accumulation in healthy organs, confirming PEA2 IgG1-FITC is capable of selective binding to CEA-expressing tumor tissue.

FIG. 6 shows the results of a quantitative biodistribution analysis of radiolabelled anti-CEA antibodies G9, F7, PEA2 in diabody format. Radiolabelled KSF antibody in diabody format was used as a negative control. The radiolabelled diabodies were injected into the lateral tail vein of BALB/c nude mice bearing subcutaneous LS174T colon adenocarcinomas. Organs were harvested 24 hours after intravenous injection and radioactivity was quantified. Results are shown as organ to blood ratios, which confirm that the G9, F7, PEA2 diabodies selectively bind to the CEA-expressing tumor tissue. In particular, these results confirm the excellent biodistribution of the PEA2 diabody, which was far superior in terms of tumor accumulation to antibodies G9 and F7, demonstrating that the biodistribution profile of antibody PEA2 is not an inherent property of antibodies which bind to the N-terminal fragment of CEA.

FIG. 7 shows the characterization of the PEA2-mIL12 and KSF-mIL12 conjugates. FIG. 7A shows the results of size exclusion chromatography of PEA2-mIL12 after purification (left) and after incubation at 37° C. for 96 hours (middle). The results of SDS-PAGE analysis of the purified PEA2-mIL12 conjugate under non-reducing (NR) and reducing (R) conditions is also shown (right). FIG. 7B shows the results of size exclusion chromatography (left) and SDS-PAGE analysis (right) of KSF-mIL12. These results confirm the purity and correct molecular weight of the PEA2-mIL12 and KSF-mIL12 conjugates. FIG. 7C shows the results of an IFN-γ release assay in which murine splenocytes were cultured for six days in a medium containing different concentrations of KSF-mIL12 and IFN-γ concentrations in the supernatant were assessed by ELISA. The results confirm the biological activity of KSF-mIL12.

FIG. 8 shows the affinity of the anti-CEA antibody conjugate PEA2-mIL12 to the N-terminal domain of CEA and the biological activity of this conjugate. FIG. 8A shows the results of BIAcore analysis using PEA2-mIL12 on a chip coated with human CEA antigen at three different concentrations of the PEA2-mIL12 conjugate: 62.5 nM, 125 nM, and 250 nM. FIG. 8B shows the results of an IFN-γ release assay in which murine splenocytes were cultured for six days in a medium containing different concentrations of PEA2-mIL12 and IFN-γ concentrations in the supernatant were assessed by ELISA. The results confirm both the binding affinity for the N-terminal domain of CEA and the biological activity of the PEA2-mIL12 conjugate.

FIG. 9 shows the results of in vivo characterization of PEA2-mIL12 in mouse tumor models. FIG. 9A shows tumor growth of CT26-CEA bearing BALB/c mice after treatment with 12 μg PEA2-mIL12, KSF-mIL12, or saline, respectively (n=3). Mice were randomized when tumors reached an average volume of 100 mm³. Arrows indicate days of injection. Error bars=SEM. FIG. 9B shows the bodyweight changes of the treated mice bearing the CT26-CEA tumors. Treatment with the PEA2-mIL12 conjugate resulted in a reduction in CT26-CEA tumor volume both in absolute terms and relative to the negative controls (KSF-mIL12; saline). Similarly, FIG. 9C shows tumor growth of C51-CEA bearing BALB/c mice after treatment with 12 μg of PEA2-mIL12, KSF-mIL12, or saline respectively (n=8). FIG. 9D shows the bodyweight change of the treated mice bearing C51-CEA tumors. Treatment with the PEA2-mIL12 conjugate resulted in C51-CEA tumor growth retardation compared to the negative controls (KSF-mIL12; saline). Together, these data confirm both the curative effect and the tolerability of PEA2-mIL12 in mouse tumor models.

FIG. 10 shows the affinity of the anti-CEA antibody conjugate PEA2-hIL2 for the N-terminal domain of CEA and the biological activity of this conjugate. FIG. 10A shows the results of size exclusion chromatography of PEA2-hIL2 after purification (left). The results of SDS-PAGE analysis of the purified PEA2-hIL2 conjugate under non-reducing (NR) and reducing (R) conditions is also shown (right). FIG. 10B shows the results of BIAcore analysis using PEA2-hIL2 on a chip coated with human CEA antigen at three different concentrations of the PEA2-mIL12 conjugate: 62.5 nM, 125 nM, and 250 nM. FIG. 10C shows the results of a proliferation assay in which murine CTLL2 cells were cultured for 96 hours in starvation medium supplemented with different concentrations of the PEA2-hIL2 conjugate. Cell survival and proliferation was quantified using the CellTiter 96™ AQueous One Solution Cell Proliferation Assay, confirming the biological activity of the PEA2-hIL2 conjugate.

FIG. 11 shows the affinity of the anti-CEA antibody conjugate PEA2-mTNF for the N-terminal domain of CEA and the biological activity of this conjugate. FIG. 11A shows the results of size exclusion chromatography of PEA2-mTNF after purification (left). The results of SDS-PAGE analysis of the purified PEA2-mTNF conjugate under non-reducing (NR) and reducing (R) conditions is also shown (right). FIG. 11B shows the results of BIAcore analysis using PEA2-mTNF on a chip coated with human CEA antigen at three different concentrations of the PEA2-mIL12 conjugate: 62.5 nM, 125 nM, and 250 nM. FIG. 11C shows the results of a proliferation assay in which murine CT26-CEA cells were cultured for 96 hours in starvation medium supplemented with different concentrations of the PEA2-mTNF conjugate. Cell survival and proliferation was quantified, using the CellTiter 96™ AQueous One Solution Cell Proliferation Assay, confirming the biological activity of the PEA2-mTNF conjugate.

FIG. 12 shows the characterisation of the PEA2-anti CD3 (PUB4) bispecific antibody in a bispecific T-cell engager format (illustrated in the first row of FIG. 12A). FIG. 12A shows the PEA2-anti-CD3 bispecific antibody in bispecific T-cell engager, scDb-scFv, IgG-scFv, IgG-(scFv)₂, scFv-IgG and scDb-scFv with Fc formats and their corresponding SEQ ID NOs. Size exclusion chromatography (FIG. 12B) and SDS-PAGE (FIG. 12C) characterization of the PEA2-PUB4 bispecific antibody in bispecific T-cell engager format confirmed its purity and correct molecular weight. FIG. 12D shows the results of flow cytometry analysis evaluating the binding of the PEA2-anti-CD3 bispecific antibody in bispecific T cell engager format to T cells and LS174T cells, which confirmed its binding to CD3 and CEA expressed on these cells. FIG. 12E shows the results of an in vitro killing assay to evaluate the activity of the PEA2-PUB4 bispecific antibody in bispecific T-cell engager format, which revealed an EC₅₀ value of 6.5 μM.

FIG. 13 shows the results of flow cytometry experiments evaluating the binding of the PEA2 antibody to transfected CHO cells transiently expressing the CEA (CEA=CEACAM5), CEACAM1, or CEACAM6 antigens. An antibody which is cross-reactive to these three antigens (clone 5B2) was used as a positive control. The results are shown in flow cytometry counterplots, plotting SSC over fluorescence intensity. The PEA2 and 5B2 antibodies both showed binding to the CEA-positive cell line, whereas only the positive control antibody 5B2 showed binding to the CEACAM1- and CEACAM6-expressing cell lines, confirming the specificity of the PEA2 antibody for CEA (CEACAM5).

FIG. 14 shows the characterisation of the PEA2-PUB4 bispecific antibody in scDb-scFv with Fc KIH and scFv-IgG4 KIH formats. FIG. 14A shows the results of size exclusion chromatography of the PEA2-PUB4 bispecific antibody in scDb-scFv with Fc KIH and scFv-IgG4 KIH formats and confirmed their purity. FIG. 14B shows the results of flow cytometry analysis evaluating the binding of the PEA2-PUB4 bispecific antibodies in scDb-scFv with Fc KIH and scFv-IgG4 KIH formats on CEA-expressing cells (MC38) and CD3 on T cells, which confirmed their binding to CD3 and CEA expressed on these cells (light grey: bispecific antibodies; dark grey: control).

FIG. 15 shows the characterisation of the anti-CD28 (AE2P)-anti-CEA (PEA2 or Sm3E) bispecific antibodies in a (2+2) IgG₄-(scFv)₂ format. FIG. 15A shows the results of size exclusion chromatography of both bispecific antibodies and confirmed their purity. FIG. 15B shows the results of flow cytometry analysis evaluating the binding of both bispecific antibodies against target and effector cells, which confirmed their binding to CD28 and CEA expressed on these cells (light grey: bispecific antibodies; dark grey: control). FIG. 15C shows the anti-CD28 IgG4 (AE2P)-anti-CEA (scFv)₂ (PEA2) bispecific antibody and the anti-CD28 IgG4 (AE2P)-anti-CEA (scFv)₂ (Sm3E) bispecific antibody formats and their corresponding SEQ ID NOS.

FIG. 16 shows the results of an in vitro activity assay using the PEA2-PUB4 bispecific antibodies and anti-CD28-anti-CEA bispecific antibodies. The percentage of target cell killing was quantitatively assessed after a 5-day co-culture of PBMCs with LS174T cells, using the PEA2-PUB4 bispecific antibodies in scDb-scFv with Fc KIH or scFv-IgG4 KIH formats, with and without the addition of anti-CD28 IgG4 (AE2P)-anti-CEA (scFv)₂ (PEA2 or Sm3E) bispecific antibody. In the combination group that targeted different CEA epitopes, the strongest in vitro lysis was observed, with the EC₅₀ value remaining unreached (FIGS. 16A and 16C). CD3⁺ T cell count was measured by flow cytometry (FIGS. 16B and 16D). The trend reflected the activity seen in the in vitro lysis assay. Data are presented as mean±SEM (n=3).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Antibody Molecule

The present invention provides antibody molecules that bind the N-terminal domain of CEA. The N-terminal domain of human CEA may comprise or consist of the sequence set forth in SEQ ID NO: 1. The antibody molecule is capable of binding to CEA expressed on the surface of a cell. Methods for determining binding of an antigen, such as CEA, are known in the art and include ELISAs and flow cytometry, for example.

The antibody molecule preferably binds CEA specifically. The term “specific” may refer to the situation in which the antibody molecule will not show any significant binding to molecules other than its specific binding partner, here CEA. For example, the antibody molecule preferably does not show any significant binding to CEACAM1 and/or CEACAM6, in particular the N-terminal domain of CEACAM1 and/or CEACAM6. Preferably, the antibody molecule does not bind CEACAM1. The term “specific” is also applicable where the antibody molecule is specific for particular epitopes, such as epitopes on CEA, that are carried by a number of antigens, in which case the antibody molecule will be able to bind to the various antigens carrying the epitope.

The antibody molecule, in scFv format, preferably binds human CEA with an affinity (K_{D}) of at least 7.7 nM. The binding affinity of an antibody molecule to a cognate antigen, such as human CEA can be determined by surface plasmon resonance (SPR), such as Biacore, e.g., as detailed in the examples.

The antibody molecule is preferably monoclonal. The antibody molecule is a human antibody molecule.

The antibody molecule may be isolated, in the sense of being free from contaminants, such as antibodies able to bind other polypeptides, and/or serum components.

The antibody molecule may be natural or partly or wholly synthetically produced. For example, the antibody molecule may be a recombinant antibody molecule.

The antibody molecule may be an immunoglobulin, or an antigen-binding fragment thereof. For example, the antibody molecule may be an IgG, IgA, IgE or IgM molecule, preferably an IgG molecule, such as an IgG1, IgG2, IgG3 or IgG4 molecule, more preferably an IgG1 or IgG4 molecule, or an antigen-binding fragment thereof.

The antigen-binding site of an antibody molecule of the invention, such as an immunoglobulin or antigen-binding fragment thereof, binds CEA. The antigen-binding site may comprise three CDRs, such as the three light chain variable domain (VL) CDRs or three heavy chain variable domain (VH) CDRs, but preferably comprises six CDRs, three VL CDRs and three VH CDRs. The three VH domain CDRs of the antigen-binding site may be located within an immunoglobulin VH domain and the three VL domain CDRs may be located within an immunoglobulin VL domain. The antibody molecule may comprise one or two antigen-binding sites for CEA. Where the antibody molecule comprises two antigen-binding sites these are preferably identical. The antibody molecule thus may comprise one VH and one VL domain but preferably comprises two VH and two VL domains, i.e. two VH/VL domain pairs, as is the case in naturally-occurring immunoglobulin molecules, scFvs, diabodies and single-chain diabodies, for example.

The antigen-binding site of the antibody molecule preferably comprises the three VL domain CDRs and/or the three VH domain CDRs of antibody PEA2. The VH and VL domain sequences of this antibody are set forth in SEQ ID NOs 8 and 9, respectively, and the sequences of the CDRs of the PEA2 antibody may be readily determined from these VH and VL domain sequences by the skilled person using routine techniques. The CDR sequences may, for example, be determined according to Kabat et al., “Sequences of Proteins of Immunological Interest,” National Institutes of Health, Bethesda, Md. (1991). In a preferred embodiment, the antigen-binding site of the antibody molecule comprises the HCDR1, HCDR2, and HCDR3 sequences set forth in SEQ ID NOs 2, 3 and 4, respectively, and the LCDR1, LCDR2 and LCDR3 sequences set forth in SEQ ID NOs 5, 6 and 7, respectively.

In a further preferred embodiment, the antigen-binding site may comprise the VH domain (SEQ ID NO: 8) and/or VL domain (SEQ ID NO: 9) of antibody PEA2, but preferably comprises the VH domain and VL domain of antibody PEA2.

The antibody molecule may also comprise a variant of a CDR, VH domain, VL domain, heavy chain or light chain sequence, as described herein. Suitable variants can be obtained by means of methods of sequence alteration, or mutation, and screening. In a preferred embodiment, an antibody molecule comprising one or more such variant sequences retain one or more of the functional characteristics of the parent antibody molecule, such as binding specificity and/or binding affinity for CEA, preferably human CEA. For example, an antibody molecule comprising one or more variant sequences preferably binds to human CEA with the same affinity as, or a higher affinity than, the (parent) antibody molecule. The parent antibody molecule is antibody molecule which does not comprise the amino acid substitution(s), deletion(s), and/or insertion(s) which has (have) been incorporated into the variant antibody molecule.

The antibody molecule may comprise a VH domain which has at least 70%, more preferably at least 75%, at least 80%, at least 85%, at least 90%, at least 95%, at least 96%, at least 97%, at least 98%, at least 99% or 100%, sequence identity to the VH domain of antibody PEA2 (SEQ ID NO: 8).

The antibody molecule may comprise a VL domain with at least 70%, more preferably one of at least 75%, at least 80%, at least 85%, at least 90%, at least 95%, at least 96%, at least 97%, at least 98%, at least 99% or 100%, sequence identity to the VL domain of antibody PEA2 (SEQ ID NO: 9).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody PEA2 in IgG1 format (SEQ ID NO: 16).

The antibody molecule may comprise a heavy chain which has at least 70%, more preferably at least 75%, at least 80%, at least 85%, at least 90%, at least 95%, at least 96%, at least 97%, at least 98%, at least 99% or 100%, sequence identity to the heavy chain of antibody PEA2 in IgG4 format (SEQ ID NO: 40).

The antibody molecule may comprise a light chain which has at least 70%, more preferably at least 75%, at least 80%, at least 85%, at least 90%, at least 95%, at least 96%, at least 97%, at least 98%, at least 99% or 100%, sequence identity to the light chain of antibody PEA2 (SEQ ID NO: 17).

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Variants of the CDRs, VH domain, VL domain, heavy chain or light chain sequence disclosed herein comprising one or more, e.g. less than 20 alterations, less than 15 alterations, less than 10 alterations or less than 5 alterations, 4, 3, 2 or 1, amino acid alterations (addition, deletion, substitution and/or insertion of an amino acid residue) may also be employed in antibody molecules according to the invention. Suitable variants can be obtained by means of methods of sequence alteration, or mutation, and screening. Alterations may be made in one or more framework regions and/or one or more CDRs. In particular, alterations may be made in HCDR1, HCDR2 and/or HCDR3, or in one or more framework regions of the heavy or light chain of the antibody molecule.

In one example, the heavy chain of an antibody molecule of the invention may comprise a C-terminal lysine residue as shown e.g. in SEQ ID NO 16, or said lysine residue may be absent. In another example, the heavy chains of the antibody molecule, in particular a bispecific antibody molecule, of the invention may comprise a knob-in-hole (KIH) mutation (Ridgway et al., 1996).

As noted above, the antibody molecule may be a whole antibody or a fragment thereof, in particular an antigen-binding fragment thereof.

Antigen-binding fragments of immunglobulins include (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al., 1989; McCafferty et al., 1990; Holt et al., 2003), which consists of a VH or a VL domain; (v) isolated CDR regions; (vi) F(ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988; Huston et al., 1988); (viii) bispecific single chain Fv dimers (WO1993/011161), (ix) “diabodies”, multivalent or multispecific fragments constructed by gene fusion (WO2013/014149; WO94/13804; Holliger et al., 1993) and (x) “single-chain diabodies” wherein two sets of VH and VL domains are connected together in sequence on the same polypeptide chain (Kontermann & Muller, 1999). Fv, scFv or diabody molecules may be stabilized by the incorporation of disulphide bridges linking the VH and VL domains (Reiter et al., 1996). Minibodies comprising a scFv joined to a CH3 domain may also be made (Hu et al., 1996). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

A single chain Fv (scFv) may be comprised within a mini-immunoglobulin or small immunoprotein (SIP), e.g. as described in (Li et al., 1997). A SIP may comprise an scFv molecule fused to the CH4 domain of the human IgE secretory isoform IgE-S2 (ε_{S2}-CH4; Batista et al., 1996), forming a homo-dimeric mini-immunoglobulin antibody molecule.

Preferably, the antibody molecule comprises or consists of a single-chain Fv (scFv), a small immunoprotein, a diabody, a single-chain diabody or a (whole) IgG molecule, such as an IgG1 or IgG4 molecule.

Where the antibody molecule is an scFv, the VH and VL domains of the antibody are preferably linked by a 14 to 20 amino acid linker. For example, the VH and VL domains may be linked by an amino acid linker which is 14, 15, 16, 17, 18, 19, or 20 amino acid in length. Suitable linker sequences are known in the art and include the linker sequence set forth in SEQ ID NO: 11.

In a preferred embodiment, the antibody molecule of the invention in scFv format comprises or consists of the sequence set forth in SEQ ID NO: 10.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen-binding site: antigen-binding sites are formed by the association of the first domain of one polypeptide within the multimer with the second domain of another polypeptide within the multimer (WO94/13804; Holliger and Winter, 1997; Holliger et al., 1993).

In a diabody or single-chain diabody, a heavy chain variable domain (VH) is connected to a light chain variable domain (VL) on the same polypeptide chain. The VH and VL domains are connected by a peptide linker that is too short to allow pairing between the two domains. This forces pairing with the complementary VH and VL domains of another chain.

Where the antibody molecule is a diabody or single-chain diabody, the VH and VL domains are preferably linked by a 5 to 12 amino acid linker. For example, the VH and VL domains may be linked by an amino acid linker which is 5, 6, 7, 8, 9, 10, 11, or 12 amino acids in length. Preferably, the amino acid linker is 5 amino acids in length. Suitable linker sequences are known in the art and include the linker sequence set forth in SEQ ID NO: 14.

In a preferred embodiment, the antibody molecule of the invention in diabody format has the sequence set forth in SEQ ID NO: 12.

In a single-chain diabody, two sets of VH and VL domains are connected together in sequence on the same polypeptide chain. For example, the two sets of VH and VL domains may be assembled in a single-chain sequence as follows: (VH-VL)-(VH-VL), where the brackets indicate a set. The two sets of VH and VL domains are connected as a single-chain by a long or ‘flexible’ peptide linker. This type of peptide linker sequence is long enough to allow pairing of the VH and VL domains of the first set with the complementary VH and VL domains of the second set. Generally, a long or ‘flexible’ linker is 15 to 20 amino acids. Suitable flexible linker sequences are known in the art and include the linker sequence set forth in SEQ ID NO: 15.

In a preferred embodiment, the antibody molecule of the invention in single-chain diabody format has the sequence set forth in SEQ ID NO: 13.

Where the antibody is a small immunoprotein (SIP) e.g. as described in (Li et al., (1997), Protein Engineering, 10:731-736), the VL domain of the scFv antibody is preferably linked to the CH4 domain of human IgE (Batista et al., (1996), J. Exp. Med., 184:2197-205) via a 2 to 20 amino acid linker, more preferably a 2 to 10 amino acid linker. Suitable linker sequences are known in the art.

In a preferred embodiment, the antibody molecule of the invention in small immunoprotein (SIP) format has the sequence set forth in SEQ ID NO: 41.

Where the antibody is a whole (complete) antibody molecule, the antibody molecule may be an IgG, IgA, IgE or IgM molecule, preferably an IgG molecule, such as an IgG1, IgG2, IgG3 or IgG4 molecule, more preferably an IgG1 or IgG4 molecule. In one preferred embodiment, the antibody molecule is an IgG1 molecule. In another preferred embodiment, the antibody molecule is an IgG4 molecule. The sequence of the PEA2 light chain in IgG format is shown in SEQ ID NO: 17, while the sequences of the PEA2 heavy chain in IgG1 format and IgG4 format are shown in SEQ ID NO: 16 and SEQ ID NO: 40 respectively.

Accordingly, in a preferred embodiment, the antibody molecule is an IgG1 molecule comprising the heavy chain amino acid sequence set forth in SEQ ID NO: 16, and the light chain amino acid sequence set forth in SEQ ID NO: 17.

In another preferred embodiment, the antibody molecule is an IgG4 molecule comprising the heavy chain amino acid sequence set forth in SEQ ID NO: 40, and the light chain amino acid sequence set forth in SEQ ID NO: 17.

### Conjugate

Conjugates of the invention comprise an antibody molecule of the invention and a therapeutic or diagnostic agent. The therapeutic agent may be a pro-inflammatory agent, a biocidal molecule, a cytotoxic molecule, a radioisotope, a photosensitizer, an enzyme, a hormone, or an immunosuppressive agent. Preferably, the therapeutic agent is a biocidal molecule, a cytotoxic molecule, a radioisotope, a pro-inflammatory agent, or an immunosuppressive agent. The biocidal molecule, cytotoxic molecule, a pro-inflammatory agent, or immunosuppressive agent may be a cytokine. In particular, the therapeutic agent conjugated to the antibody molecule may have immunosuppressive activity. Most preferably, the therapeutic agent conjugated to the antibody molecule of the invention is a pro-inflammatory agent, in particular a pro-inflammatory cytokine.

Pro-inflammatory cytokines which may be conjugated to an antibody molecule of the invention include interleukin-2 (IL2), interleukin-12 (IL12), and tumour necrosis factor (TNF), such as TNFα, as well as mutants or variants thereof. Preferably, the cytokine conjugated to an antibody molecule of the invention is IL2, IL12, or TNFα, as well as mutants or variants thereof. The TNFα mutant or variant may retain the cytotoxic and/or pro-inflammatory activities of wild type (wt) TNFα. The TNFα mutant or variant may have reduced cytotoxic and/or pro-inflammatory activities relative to wild type (wt) TNFα. The sequence of human IL2 (IL2) is set forth in SEQ ID NO: 23. The sequence of human single-chain IL12 as disclosed in WO2013/014149 is set forth in SEQ ID NO: 27. The sequence of the soluble form of the extracellular domain of human TNFα, which may be conjugated to an antibody molecule of the invention, is set forth in SEQ ID NO: 29. The sequence of the soluble form of the extracellular domain of human TNFα mutant (R32A), which may be conjugated to an antibody molecule of the invention, is set forth in SEQ ID NO: 32. The sequences of the remaining cytokines, as well as variants thereof which may be employed in the present invention, are known in the art.

A therapeutic agent may be conjugated to the N-terminus or C-terminus of the antibody molecule or both. Where a therapeutic agent is conjugated to both the N-terminus and the C-terminus of the antibody molecule, the therapeutic agents may be the same or different but preferably are different. Where the therapeutic agent is conjugated to the N-terminus of the antibody molecule, the C-terminus may be “free”, i.e. not conjugated to another moiety. Similarly, where the therapeutic agent is conjugated to the C-terminus of the antibody molecule, the N-terminus may be “free”, i.e., not conjugated to another moiety.

In a preferred embodiment, the antibody molecule, preferably in single-chain diabody format, is conjugated to interleukin 12 (IL12). In a preferred embodiment, the antibody molecule is conjugated at its N-terminus to IL12. In a more preferred embodiment, the conjugate comprises or consists of the sequence set forth in SEQ ID NO: 26.

In another preferred embodiment, the antibody molecule, preferably in diabody format, is conjugated to interleukin 2 (IL2). In a preferred embodiment, the antibody molecule is conjugated at its C-terminus to IL2. In a more preferred embodiment, the conjugate comprises or consists of the sequence set forth in SEQ ID NO 21.

In a yet further preferred embodiment, the antibody molecule, preferably in scFv format, is conjugated, preferably at its C-terminus, to a mutant of tumour necrosis factor alpha (TNFα). In a more preferred embodiment, the conjugate comprises or consists of the sequence set forth in SEQ ID NO: 28.

In another preferred embodiment, the antibody molecule, preferably in scFv format, is conjugated, preferably at its N-terminus, to interleukin 2 (IL2) and, preferably at its C-terminus, to a mutant of tumour necrosis factor alpha (TNFα). In a more preferred embodiment, the conjugate comprises or consists of the sequence set forth in SEQ ID NO: 30.

A diagnostic agent conjugated to the antibody molecule of the invention may be a detectable label, such as a radioisotope, e.g. a non-therapeutic radioisotope.

Radioisotopes which may be conjugated to an antibody molecule of the invention include isotopes such as ^{94m}Tc, ^{99m}Tc, ¹⁸⁶Re, ¹⁸⁸Re, ²⁰³Pb, ⁶⁷Ga, ⁶⁸Ga, ⁴⁷Sc, ¹¹¹In, ⁹⁷Ru, ⁶²Cu, ⁶⁴Cu, ⁸⁶Y, ⁸⁸Y, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At and ²²⁵Ac. Preferably, positron emitters, such as ¹⁸F and ¹²⁴I, or gamma emitters, such as ^{99m}Tc, ¹¹¹In and ¹²³I, are used for diagnostic applications (e.g. for PET), while beta-emitters, such as ¹³¹I, ⁹⁰Y and ¹⁷⁷Lu, are preferably used for therapeutic applications. Alpha-emitters, such as ²¹¹At and ²²⁵Ac may also be used for therapy. In one example, the antibody molecule may be conjugated to ¹⁷⁷Lu, ¹³¹I, or ⁹⁰Y.

The antibody molecule may be conjugated with the therapeutic agent by means of a peptide bond or linker as described herein. Other means for conjugation include chemical conjugation, especially cross-linking using a bifunctional reagent (e.g. employing DOUBLE-REAGENTS™ Cross-linking Reagents Selection Guide, Pierce).

### Bispecific Binding Molecules

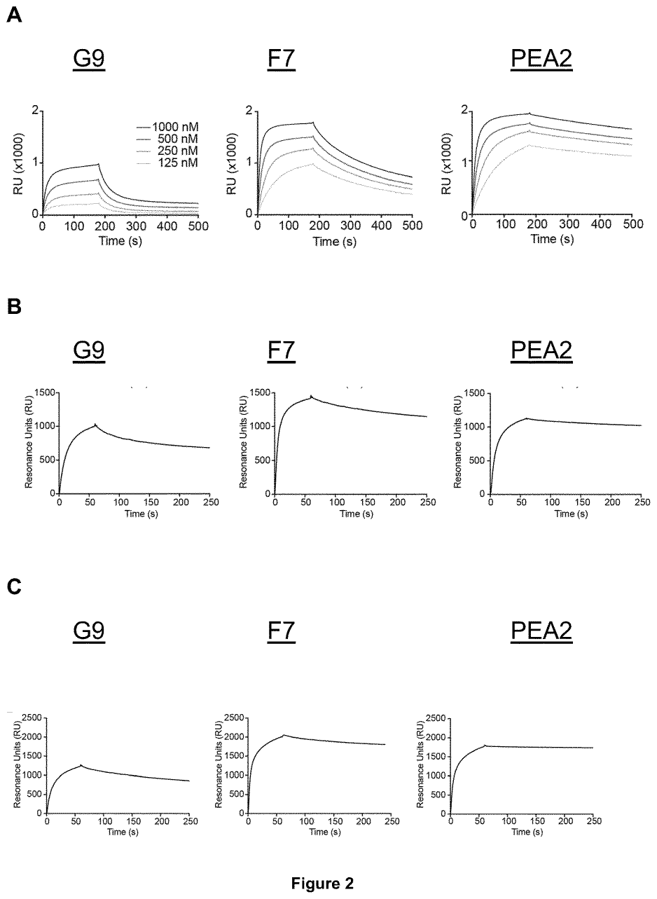

The antibody molecule of the present invention may be a bispecific antibody molecule comprising a second antigen-binding site for a second target antigen. The second target antigen is preferably not CEA. Examples of suitable bispecific antibody molecules for use in the context of the present invention can be found in Kontermann 2012 (page 186 FIG. 2) the content of which is incorporated herein by reference.

Examples of preferred bispecific antibody molecules are IgG-appended antibodies with an additional antigen-binding moiety, preferably an scFv, conjugated to the C-terminus of the antibody molecule. Examples of such antibody molecules are IgG-(scFv)₂ and IgG-scFv.

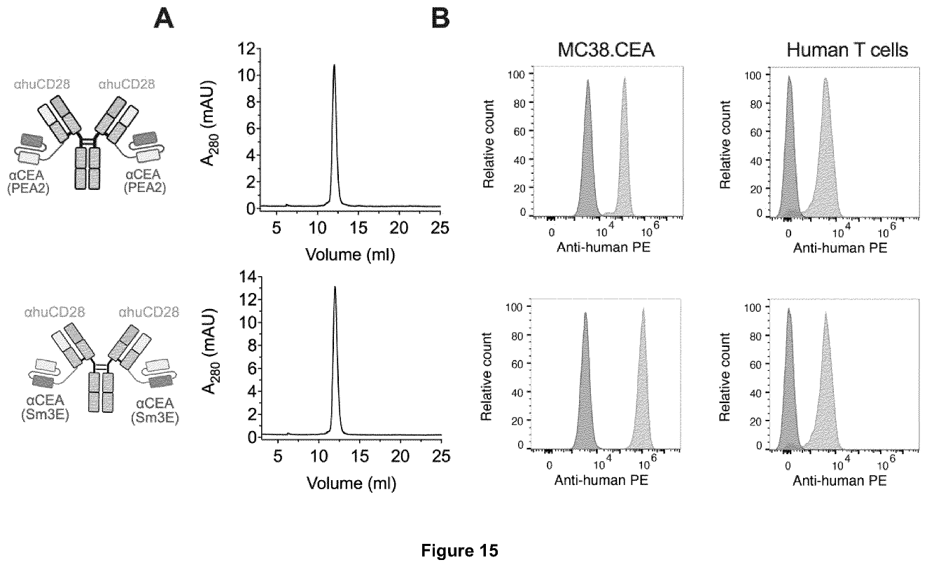

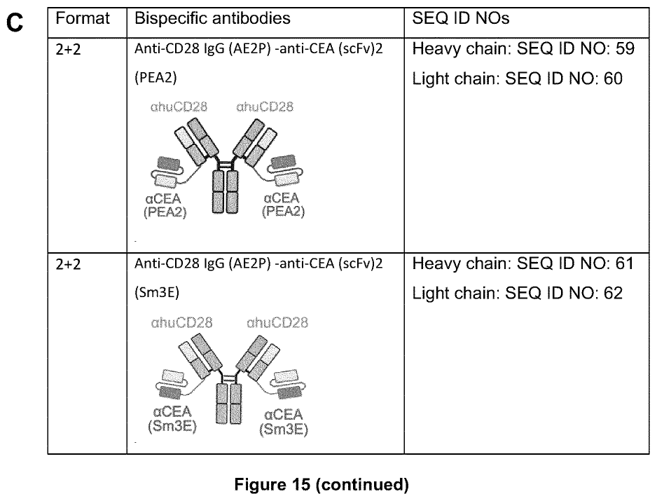

Other examples of preferred bispecific antibody molecules are IgG-appended antibodies with an additional antigen-binding moiety, preferably an scFv, conjugated to the N-terminus of the antibody molecule. In particular, the scFv may be conjugated to the N-terminus of one heavy chain of the antibody molecule, with the N-terminus of the other heavy chain being free, i.e. unconjugated. An example of such an antibody molecule is an scFv-IgG KIH. Further examples of preferred bispecific antibody molecules are IgG-appended antibodies with an additional antigen-binding moiety, preferably an scFv, conjugated to the C-terminus of the CL domain of the antibody molecule. An example of such an antibody molecule is an IgG-(scFv)₂ as shown in FIG. 15C, for example. Other preferred bispecific antibody molecules are small recombinant bispecific antibody formats, such as bispecific T-cell engager, scDb-scFv and scDb-scFv with Fc KIH, e.g. bispecific T-cell engager and scDb-scFv.

The bispecific binding molecule of the invention preferably comprises a second antigen-binding site which binds a T cell antigen, most preferably CD3. Where the bispecific binding molecule comprises an antigen-binding site for CD3, the antigen-binding site may comprise the VH domain or VL domain sequence, but preferably comprises the VH domain and VL domain sequence, of the anti-CD3 antibody molecule set forth in SEQ ID NOs: 51 and 52, respectively. In a preferred embodiment, the antigen-binding site for CD3 is provided by an scFv conjugated at the N- or C-terminus to the antibody molecule of the invention to provide a bispecific binding molecule. The anti-CD3 antibody in scFv format preferably has the sequence set forth in SEQ ID NO: 53.

In one preferred embodiment, the bispecific antibody molecule is a bispecific T cell engager molecule comprising the PEA2 antibody in scFv format and an anti-CD3 in scFv format. The sequence of such a bispecific antibody molecule is set forth in SEQ ID NO: 33.

In another preferred embodiment, the bispecific antibody molecule is an scDb-scFv and comprises the PEA2 antibody in scDb format and an anti-CD3 in scFv format. The sequence of such a bispecific antibody molecule is set forth in SEQ ID NO: 34.

Where the bispecific antibody molecule is in IgG-scFv format, the antibody molecule preferably comprises a “knob-in-hole” (KIH) mutation pair to facilitate assembly of the two different heavy chains in the correct heavy chain pair. Knob-in-hole mutations are well known in the art, as is their use in the preparation of bispecific antibody molecules. Specifically, the first heavy chain comprises a “knob” mutation and the second heavy chain a “hole” mutation to drive assembly of the two heavy chains. The IgG-scFv preferably comprises a common light chain.

The sequence of a “knob” mutation-containing IgG4 heavy chain of the PEA2 antibody conjugated at its C-terminus to an anti-CD3 scFv is shown in SEQ ID NO: 48. The sequence of a “hole” mutation-containing IgG4 heavy chain of the PEA2 antibody is shown in SEQ ID NO: 49.

The heavy chain sequence of a bispecific antibody molecule comprising the PEA2 antibody in IgG4 format and two anti-CD3 scFvs conjugated to the C-termini of the two heavy chains to provide an IgG-(scFv)₂ is shown in SEQ ID NO: 50.

The sequence of the PEA2 light chain is shown in SEQ ID NO: 17.

Accordingly, in a preferred embodiment, the bispecific antibody molecule is an IgG4-scFv molecule comprising the heavy chain amino acid sequences set forth in SEQ ID NOs: 48 and 49, and the light chain amino acid sequence set forth in SEQ ID NO: 17.

In another preferred embodiment, the bispecific antibody molecule is an IgG4-(scFv)₂ molecule comprising the heavy chain amino acid sequence set forth in SEQ ID NO: 50, and the light chain amino acid sequence set forth in SEQ ID NO: 17.

In another preferred embodiment, the bispecific antibody molecule is a scDb-scFv with Fc KIH molecule comprising the PEA2 antibody in scDb format and an anti-CD3 antibody in scFv format. The sequence of such a bispecific antibody molecule is set forth in SEQ ID NOs: 54 and 55.

In another preferred embodiment, the bispecific antibody molecule is a scFv-IgG KIH molecule comprising the PEA2 antibody in IgG4 KIH format and an anti-CD3 antibody in scFv format. The sequence of such a bispecific antibody molecule is set forth in SEQ ID NOs: 56, 57 and 58.

In one preferred embodiment, the bispecific antibody molecule is an IgG-(scFv)₂ molecule comprising the PEA2 antibody in scFv format and an anti-CD28 in IgG4 format. The sequence of such a bispecific antibody molecule is set forth in SEQ ID NOs: 59 and 60.

### Linkers

The antibody molecule, e.g. scFv or IgG, and the therapeutic or diagnostic agent or molecule may be connected to each other directly, for example through any suitable chemical bond, but preferably are connected via a peptide linker. The chemical bond may be, for example, a covalent or ionic bond. Examples of covalent bonds include peptide bonds (amide bonds) and disulphide bonds.

Where the therapeutic or diagnostic agent is connected to the antibody molecule via a peptide linker, the peptide linker may be a short (2-30, preferably 5-20) residue stretch of amino acids. Suitable examples of peptide linker sequences are known in the art. One or more different linkers may be used. Exemplary linkers are set forth in SEQ ID NOs 11, 14, 15, 19, 22, 31 and 35-37, for example. In one embodiment, the linker may be a cleavable linker.

Where the antibody molecule and therapeutic or diagnostic agent are connected via a peptide bond or peptide linker, the conjugate may be produced (secreted) as a single chain polypeptide, such as a fusion protein.

### Methods of Treatment

As explained above, the presence of CEA and cells expressing CEA on their surface has been shown to be associated with a number of diseases and disorders, including cancer.

An antibody molecule or conjugate of the invention may therefore be for use as a medicament.

In particular, the antibody molecule or conjugate may be for use in a method of treatment (which may include prophylactic treatment) of the human or animal body.

Also provided is a method of treating a disease or disorder in a patient, wherein the method comprises administering to the patient a therapeutically effective amount of the antibody molecule or conjugate.

Further provided is the use of the antibody molecule or conjugate in the manufacture of a medicament for use in the treatment of a disease or disorder in a patient.

The patient may be a human patient or may be an animal patient.

Treatment may be any treatment or therapy in which some desired therapeutic effect is achieved, for example, the inhibition or delay of the progress of the disease or disorder, and includes a reduction in the rate of progress, a halt in the rate of progress, amelioration of the disease or disorder, cure or remission (whether partial or total) of the disease or disorder, preventing, ameliorating, delaying, abating or arresting one or more symptoms and/or signs of the disease or disorder or prolonging survival of an individual or patient beyond that expected in the absence of treatment.

Treatment as a prophylactic measure (i.e. prophylaxis) is also included. For example, an individual susceptible to or at risk of the occurrence or re-occurrence of a disease or disorder may be treated as described herein. Such treatment may prevent or delay the occurrence or re-occurrence of the disease or disorder in the individual.

A method of treatment as described may comprise administering at least one further treatment to the individual in addition to the antibody molecule or conjugate. The antibody molecule or conjugate may thus be administered to an individual alone or in combination with one or more other treatments for the disease or disorder in question. Where the antibody molecule or conjugate is administered to the individual in combination with another treatment, the additional treatment may be administered to the individual concurrently with, sequentially to, or separately from the administration of the antibody molecule or conjugate. Where the additional treatment is administered concurrently with the antibody molecule or conjugate, the antibody molecule or conjugate and additional treatment may be administered to the patient as a combined preparation. For example, the additional therapy may be a known therapy or therapeutic agent for the disease or disorder to be treated.

In a preferred embodiment, the antibody or conjugate of the invention is administered to a patient in combination with a therapeutic agent, such as chemotherapy, radiation therapy, an immunomodulatory agent, an immunoconjugate such as an immunocytokine, or a bispecific antibody.

The present invention thus provides an antibody or conjugate of the invention for use in a method of treating cancer in a patient, wherein the method further comprises administering a therapeutic agent to the patient. Also provided is a method of treating cancer in a patient, wherein the method comprises administering an antibody or conjugate of the invention and a therapeutic agent to the patient. The therapeutic agent may be administered to the patient concurrently with, sequentially to, or separately from the administration of the antibody or conjugate. Further provided is the use of an antibody or conjugate of the invention for the manufacture of a medicament for treating cancer, wherein treatment comprises administering the antibody or conjugate and a therapeutic agent to the patient.

In a preferred embodiment, a bispecific antibody molecule which binds CEA and T cell antigen may be administered in combination with a bispecific antibody molecule of the invention. In this context, the bispecific antibody molecule of the invention preferably binds CEA and CD3. The T cell antigen bound by the second bispecific antibody molecule is preferably not CD3. In a preferred embodiment, the T cell antigen bound by the second bispecific antibody molecule is CD28. Thus, a bispecific antibody molecule which binds CEA and CD28 may be administered in combination with a bispecific antibody molecule of the invention which binds CEA and CD3. The anti-CEA antigen binding site of the bispecific anti-CEA anti-CD28 antibody molecule may bind the same epitope as the PEA2 antibody of the invention, or a different epitope, but preferably binds a different epitope on CEA than the PEA2 antibody of the invention.

Where the anti-CEA anti-CD28 antibody molecule binds the same epitope as the PEA2 antibody of the invention, the anti-CEA anti-CD28 antibody molecule may comprise the HCDR1, HCDR2, and HCDR3 and LCDR1, LCDR2 and LCDR3 sequences, and/or VH and VL sequences of the PEA2 antibody described herein.

Where the anti-CEA anti-CD28 antibody molecule binds a different epitope on CEA than the PEA2 antibody of the invention, the antibody molecule may comprise the HCDR1, HCDR2, and HCDR3 and LCDR1, LCDR2 and LCDR3 sequences, and/or VH and VL sequences of the anti-CEA antibody Sm3E shown herein, e.g. in the antibody molecule comprising SEQ ID NOs 61 and 62.

An example of an anti-CD28 antibody (AE2P) which may be incorporated into an anti-CEA anti-CD28 antibody molecule is described in PCT/EP2023/070320.

In one preferred embodiment, a bispecific antibody molecule of the invention binding CEA and CD3 in a scDb-scFv with Fc KIH format is administered in combination with an IgG-(scFv)₂ molecule comprising an anti-CD28 antibody in IgG4 format and anti-CEA antibody of the invention (PEA2) in scFv format. In a preferred example, a bispecific antibody molecule comprising, or consisting of, the sequences set forth in SEQ ID NOs: 54 and 55 is administered in combination with a bispecific antibody molecule comprising, or consisting of, the sequences set forth in SEQ ID NOs: 59 and 60.

In another preferred embodiment, a bispecific antibody molecule of the invention binding CEA and CD3 in a scDb-scFv with Fc KIH format is administered in combination with an IgG-(scFv)₂ molecule comprising an anti-CD28 antibody in IgG₄ format and an anti-CEA antibody other than the antibody of the invention in scFv format (e.g. Sm3E). In a preferred example, a bispecific antibody molecule comprising, or consisting of, the sequences set forth in SEQ ID NOs: 54 and 55 is administered in combination with a bispecific antibody molecule comprising, or consisting of, the sequences set forth in SEQ ID NOs: 61 and 62.

In another preferred embodiment, a bispecific antibody molecule of the invention binding CEA and CD3 in a scFv-IgG KIH format is administered in combination with an IgG-(scFv)₂ molecule comprising an anti-CD28 antibody in IgG4 format and anti-CEA antibody of the invention (PEA2) in scFv format. In a preferred example, a bispecific antibody molecule comprising, or consisting of, the sequences set forth in in SEQ ID NOs: 56, 57 and 58 is administered in combination with a bispecific antibody molecule comprising, or consisting of, the sequences set forth in SEQ ID NOs: 59 and 60.

In another preferred embodiment, a bispecific antibody molecule of the invention binding CEA and CD3 in a scFv-IgG KIH format is administered in combination with an IgG-(scFv)₂ molecule comprising an anti-CD28 antibody in IgG₄ format and an anti-CEA antibody other than the antibody of the invention (e.g. Sm3E) in scFv format. In a preferred example, a bispecific antibody molecule comprising, or consisting of, the sequences set forth in SEQ ID NOs: 56, 57 and 58 is administered in combination with a bispecific antibody molecule comprising, or consisting of, the sequences set forth in SEQ ID NOs: 61 and 62. In another preferred embodiment, the therapeutic agent is selected from the group consisting of: chemotherapy, radiation therapy, immunomodulatory agent, an immunoconjugate such as an immunocytokine, or a bispecific antibody.

In a further preferred embodiment, the antibody or conjugate of the invention is administered to a patient in combination with an immunomodulatory agent, such as an anti-PD-1 antibody, anti-PD-L1 antibody, anti-LAG-3 antibody, anti-TIGIT antibody, or anti-TIM-3 antibody. Most preferably, the immunomodulatory agent is an anti-PD-1 antibody. The present invention thus provides an antibody or conjugate of the invention for use in a method of treating cancer in a patient, wherein the method further comprises administering an immunomodulatory agent to the patient. Also provided is a method of treating a cancer in a patient, wherein the method comprises administering an antibody or conjugate of the invention and an immunomodulatory agent to the patient. The immunomodulatory agent may be administered to the patient concurrently with, sequentially to, or separately from the administration of the antibody or conjugate. Further provided is the use of an antibody or conjugate of the invention for the manufacture of a medicament for the treatment of cancer, wherein treatment comprises administering the antibody or conjugate and an immunomodulatory agent to the patient.

Anti-PD-1, anti-PD-L1, anti-LAG-3, anti-TIGIT, and anti-TIM-3 antibodies are known in the art and are available to the skilled person. A number of anti-PD-1 and anti-PDL-1 antibodies are licensed for the treatment of cancer in human patients and can be employed in treatment of cancer in a patient in combination with an antibody or conjugate of the invention.

The antibody administered to the patient in combination with an immunomodulatory agent preferably is a bispecific antibody molecule as disclosed herein, such as a bispecific antibody molecule in IgG-(scFv)₂, IgG-scFv, bispecific T-cell engager, or scDb-scFv format. Most preferably the bispecific antibody administered to the patient in combination with an immunomodulatory agent is in scDb-scFv format and comprises, or consists of, the sequence set forth in SEQ ID NO: 34.

The immunomodulatory agent administered to the patient in combination with a bispecific antibody of the present invention preferably is an anti-PD-1 antibody.

Accordingly, in a preferred embodiment, the antibody administered to the patient in combination with an anti-PD-1 antibody is a bispecific T-cell engager comprising the PEA2 antibody in scFv format and an anti-CD3 in scFv format. The sequence of such a bispecific antibody molecule is set forth in SEQ ID NO: 33.

In another preferred embodiment, the antibody administered to the patient in combination with an anti-PD-1 antibody is an scDb-scFv and comprises the PEA2 antibody in scDb format and an anti-CD3 in scFv format. The sequence of such a bispecific antibody molecule is set forth in SEQ ID NO: 34.

In a further preferred embodiment, the antibody administered to the patient in combination with an anti-PD-1 antibody is an IgG4-scFv molecule comprising the heavy chain amino acid sequences set forth in SEQ ID NOS: 48 and 49, and the light chain amino acid sequence set forth in SEQ ID NO: 17.

In yet another preferred embodiment, the antibody administered to the patient in combination with an anti-PD-1 antibody is an IgG4-(scFv)₂ molecule comprising the heavy chain amino acid sequence set forth in SEQ ID NO: 50, and the light chain amino acid sequence set forth in SEQ ID NO: 17.

The conjugate administered to the patient in combination with an immunomodulatory agent preferably is a conjugate as disclosed herein. More preferably, the conjugate administered to the patient in combination with an immunomodulatory agent or a bispecific antibody comprises a single-chain diabody conjugated at its N-terminus to IL12, wherein the conjugate comprises the CDRs and/or VH and VL domains of the PEA2 anti-CEA antibody, as disclosed herein. Most preferably the conjugate comprises, or consists of, the sequence set forth in SEQ ID NO: 26.

The immunomodulatory agent administered to the patient in combination with a conjugate of the present invention preferably is an anti-PD-1 antibody.

Accordingly, in a preferred embodiment, the conjugate administered to the patient in combination with an anti-PD-1 antibody comprises a single-chain diabody conjugated at its N-terminus to IL12, wherein the conjugate comprises the CDRs and/or VH and VL domains of the PEA2 anti-CEA antibody, as disclosed herein. The sequence of such a conjugate is set forth in SEQ ID NO: 26.

The disease or disorder to be treated may be a disease or disorder characterised by the expression or overexpression of CEA on the surface of cells, such as cancer cells.

The disease or disorder to be treated using an antibody molecule or conjugate of the invention may be any disease or disorder characterised by, or associated with, the expression of CEA. As explained above, in healthy individuals, expression of CEA is restricted to the apical surface of mature enterocytes and is inaccessible for antibodies in circulation. However, in most colorectal cancer tissues and various other malignancies, CEA becomes heavily overexpressed on the whole surface of tumor cells and consequently becomes exposed to the vasculature and lymphatic system. This selective accessibility means CEA is expected to represent a disease-specific target for therapy in diseases or disorders characterised by expression of CEA. For example, the disease or disorder may be characterised by, or associated with, the presence of CEA on cell surfaces, as is known to be the case for a wide variety of cancers. In addition, or alternatively, the disease to be treated may be cancer, wherein the cancer cells express CEA. As a further alternative, the disease or disorder may be associated with, or characterised by, the presence of tissue comprising expression of CEA, for example as a result of the presence of cells expressing CEA on their surface.

The disease to be treated using an antibody molecule or conjugate of the invention may be cancer, as well as other tumours and neoplastic conditions.

Exemplary cancers include any type of solid or non-solid cancer or malignant lymphoma and especially liver cancer, lymphoma, leukaemia (e.g. acute myeloid leukaemia), sarcomas, skin cancer, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic. The cancer, tumour, or neoplastic condition may express CEA or comprise CEA-expressing cells, for example cells that express CEA on their cell surface. In a preferred embodiment, the cancer is colorectal cancer, lung cancer, stomach cancer or pancreatic cancer.

### Methods of Detection or Diagnosis

The antibody molecules and conjugates are suitable for detecting CEA in vivo and in vitro, and thus find application in the imaging, detection and diagnosis of disease characterised by, or associated with, expression of CEA, e.g., as the result of the presence of cells expressing CEA on their cell surface.

The present invention therefore also relates to the use of an antibody molecule or conjugate of the invention for detecting CEA, e.g., cells expressing CEA on their cell surface, either in vitro or in vivo. The conjugate preferably comprises a detectable label to aid detection. The preparation of suitable conjugates is described elsewhere herein. Alternatively, binding of the antibody molecule to CEA may be detected using a secondary antibody or other detection reagent. Where the antibody molecule is conjugated to a radioisotope, binding of the antibody molecule to CEA in the patient may be detected using scintigraphy.

Also provided is an in vitro method for detecting CEA, the method comprising incubating the antibody molecule or conjugate with a sample obtained from an individual, e.g., a human patient, and detecting binding of the antibody molecule or conjugate to the sample, e.g., cells present in the sample, wherein binding of the antibody molecule or conjugate to the sample indicates the presence of CEA. Methods for determining binding of an antibody molecule or antigen to a sample are known in the art and include, for example, ELISAs, flow cytometry, and immunostaining of tissue samples.

Further provided is the antibody molecule or conjugate for use in a method of detecting CEA in vivo, the method comprising administering the antibody molecule or conjugate to an individual, e.g., a human patient, wherein localisation of the antibody molecule or conjugate at a site in the individual, indicates expression of CEA at said site.

In healthy individuals, expression of CEA is inaccessible for antibodies in circulation, but CEA is known to be overexpressed on the whole surface of tumor cells and consequently becomes exposed to the vasculature and lymphatic system, the antibody molecules and conjugates of the invention are also expected to find application in the detection of diseases and disorders characterised by expression of CEA. Thus, the present invention also provides an antibody molecule or conjugate of the invention for use a detection agent, diagnostic, or imaging agent.

Thus, the present invention also provides the antibody molecule or conjugate for use in a method of imaging, detecting, or diagnosing a disease or disorder in a patient.

Also provided is a method of imaging, detecting, or diagnosing a disease or disorder in a patient comprising administering an antibody molecule or conjugate of the invention to the patient.

Further provided is the use of an antibody molecule or conjugate of the invention in the manufacture of a diagnostic product for use in the detection or diagnosis of a disease or disorder.

The disease or disorder is preferably characterised by expression of CEA, such as the presence of CEA-expressing cells, and may be a disease or disorder as described herein, such as cancer.

### Pharmaceutical Compositions

Whilst an antibody molecule or conjugate may be administered alone, antibody molecules and conjugates will typically be administered in the form of a pharmaceutical composition. Thus, a further aspect of the present invention relates to a pharmaceutical composition comprising at least one antibody molecule or conjugate of the invention and at least one other component, such as a pharmaceutically acceptable excipient. A method comprising formulating an antibody molecule or conjugate into a pharmaceutical composition is also provided.

Pharmaceutical compositions may comprise, in addition to the antibody molecule or conjugate, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g. by injection, the pharmaceutical composition comprising the antibody molecule or conjugate may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required, including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagine, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

In some embodiments, the antibody molecules or conjugates may be provided in a lyophilised form for reconstitution prior to administration. For example, lyophilised antibody molecules or conjugates may be re-constituted in sterile water and mixed with saline prior to administration to an individual.

Administration may be in a “therapeutically effective amount”, this being sufficient to show benefit to an individual. The actual amount administered, and rate and time-course of administration, will depend on the nature and severity of the disease or disorder being treated, the particular individual being treated, the clinical condition of the individual, the cause of the disorder, the site of delivery of the composition, the type of antibody molecule or conjugate, the method of administration, the scheduling of administration and other factors known to medical practitioners. Prescription of treatment, e.g. decisions on dosage etc., is within the responsibility of general practitioners and other medical doctors. Appropriate doses of antibody molecules are well known in the art (Ledermann et al., 1991; Bagshawe et al., 1991). Specific dosages indicated herein, or in the Physician's Desk Reference (2003) as appropriate for an antibody molecule being administered, may be used. Appropriate doses for conjugates are also known or can be determined. For example, a therapeutically effective amount or suitable dose of an antibody molecule or conjugate can be determined by comparing in vitro activity and in vivo activity in an animal model. Methods for extrapolation of effective dosages in domestic dogs, pigs and sheep, as well as other test animals to humans are known. The precise dose will depend upon a number of factors, including whether the size and location of the area to be treated, and the precise nature of the antibody molecule or conjugate.

Treatments may be repeated at daily, twice-weekly, weekly or monthly intervals, at the discretion of the physician. The treatment schedule for an individual may be dependent on the pharmacokinetic and pharmacodynamic properties of the antibody molecule or conjugate, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Suitable formulations and routes of administration are described above.

A pharmaceutical composition may be administered alone or in combination with other treatments, either simultaneously or sequentially dependent upon the condition to be treated.

### Kits

Another aspect of the invention provides a therapeutic kit for use in the treatment of a disease or disorder comprising an antibody molecule or conjugate as described herein. The components of a kit are preferably sterile and in sealed vials or other containers.

A kit may further comprise instructions for use of the components in a method described herein. The components of the kit may be comprised or packaged in a container, for example a bag, box, jar, tin or blister pack.

In a preferred embodiment, the kit comprises a bispecific antibody molecule which binds CEA and T cell antigen and a bispecific antibody molecule of the invention which binds CEA and CD3. The T cell antigen in this context is preferably not CD3. In a preferred embodiment, the T cell antigen is CD28. Thus, provided is a kit comprising a bispecific antibody molecule which binds CEA and CD28 and a bispecific antibody molecule of the invention which binds CEA and CD3. The anti-CEA antigen binding site of the bispecific anti-CEA anti-CD28 antibody molecule may bind the same epitope as the PEA2 antibody of the invention, or a different epitope, but preferably binds a different epitope on CEA than the PEA2 antibody of the invention.

Where the anti-CEA anti-CD28 antibody molecule binds the same epitope as the PEA2 antibody of the invention, the anti-CEA anti-CD28 antibody molecule may comprise the HCDR1, HCDR2, and HCDR3 and LCDR1, LCDR2 and LCDR3 sequences, and/or VH and VL sequences of the PEA2 antibody described herein.

Where the anti-CEA anti-CD28 antibody molecule binds a different epitope on CEA than the PEA2 antibody of the invention, the antibody molecule may comprise the HCDR1, HCDR2, and HCDR3 and LCDR1, LCDR2 and LCDR3 sequences, and/or VH and VL sequences of the anti-CEA antibody Sm3E shown herein, e.g. in the antibody molecule comprising SEQ ID NOs 61 and 62.

An example of an anti-CD28 antibody (AE2P) which may be incorporated into an anti-CEA anti-CD28 antibody molecule is described in PCT/EP2023/070320.

In a preferred embodiment, the kit comprises a bispecific antibody molecule of the invention which binds CEA and CD3 in a scDb-scFv with Fc KIH format and an IgG-(scFv)₂ molecule comprising an anti-CD28 antibody in IgG4 format and anti-CEA antibody of the invention (PEA2) in scFv format. In a preferred example, the kit comprises a bispecific antibody molecule comprising, or consisting of, the sequences set forth in SEQ ID NOs: 54 and 55 and a bispecific antibody molecule comprising, or consisting of, the sequences set forth in SEQ ID NOs: 59 and 60.

In another preferred embodiment, the kit comprises a bispecific antibody molecule of the invention binding CEA and CD3 in a scDb-scFv with Fc KIH format and an IgG-(scFv)₂ molecule comprising an anti-CD28 antibody in IgG4 format and an anti-CEA antibody other than the antibody of the invention in scFv format (e.g. Sm3E). In a preferred example, the kit comprises a bispecific antibody molecule comprising, or consisting of, the sequences set forth in SEQ ID NOs: 54 and 55 and a bispecific antibody molecule comprising, or consisting of, the sequences set forth in SEQ ID NOs: 61 and 62.

In another preferred embodiment, the kit comprises a bispecific antibody molecule of the invention binding CEA and CD3 in a scFv-IgG KIH format and an IgG-(scFv)₂ molecule comprising an anti-CD28 antibody in IgG4 format and anti-CEA antibody of the invention (PEA2) in scFv format. In a preferred example, the kit comprises a bispecific antibody molecule comprising, or consisting of, the sequences set forth in in SEQ ID NOs: 56, 57 and 58 and a bispecific antibody molecule comprising, or consisting of, the sequences set forth in SEQ ID NOs: 59 and 60.

In another preferred embodiment, the kit comprises a bispecific antibody molecule of the invention binding CEA and CD3 in a scFv-IgG KIH format and an IgG-(scFv)₂ molecule comprising an anti-CD28 antibody in IgG4 format and an anti-CEA antibody other than the antibody of the invention (e.g. Sm3E) in scFv format. In a preferred example, the kit comprises a bispecific antibody molecule comprising, or consisting of, the sequences set forth in SEQ ID NOs: 56, 57 and 58 and a bispecific antibody molecule comprising, or consisting of, the sequences set forth in SEQ ID NOs: 61 and 62.

### Nucleic Acids, Vectors, Host Cells, and Methods of Production

Provided is an isolated nucleic acid molecule encoding an antibody molecule or conjugate of the invention. Nucleic acid molecules may comprise DNA and/or RNA and may be partially or wholly synthetic.

An isolated nucleic acid molecule may be used to express an antibody molecule or conjugate of the invention. The nucleic acid will generally be provided in the form of an expression vector. Another aspect of the invention thus provides an expression vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g., phage, or phagemid, as appropriate in the context.

A nucleic acid molecule or expression vector as described herein may be introduced into a host cell. Techniques for the introduction of nucleic acid or vectors into host cells are well established in the art and any suitable technique may be employed. A range of host cells suitable for the production of recombinant antibody molecules and conjugates are known in the art, and include bacterial, yeast, insect or mammalian host cells. A preferred host cell is a mammalian cell, such as a CHO, NS0, or HEK cell, for example a HEK293 cell.

Another aspect of the invention provides a method of producing an antibody molecule, or conjugate, comprising expressing a nucleic acid encoding the antibody molecule, or conjugate, in a host cell and optionally isolating and/or purifying the antibody molecule, or conjugate, thus produced. Methods for culturing host cells are well-known in the art. The method may further comprise isolating and/or purifying the antibody molecule or conjugate. Techniques for the purification of recombinant antibody molecules, or conjugates, are well-known in the art and include, for example HPLC, FPLC, or affinity chromatography, e.g., using Protein A or Protein L. In some embodiments, purification may be performed using an affinity tag on antibody molecule. The method may also comprise formulating the antibody molecule, or conjugate, into a pharmaceutical composition, optionally with a pharmaceutically acceptable excipient or other substance as described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1: Cloning of N-Terminal Domain of Carcinoembryonic Antigen (CEA) Including Characterization, Phage Display Selection and Affinity Maturation Against Antigen, and Isolation of G9, F7 And PEA2 Antibodies in scFv Format

### 1.1 Expression Procedure

A human CEA (N-terminal domain) recombinant fragment containing a C-terminal 6×His tag and a N-terminal BirA target sequence (GLNDIFEAQKIEWHE, SEQ ID NO: 37) was expressed by transient gene expression (TGE) in CHO-S cells using Polyethyleneimine (PEI). The protein fragment was purified from the cell culture medium by nickel affinity chromatography and then dialyzed into phosphate buffered saline (PBS, pH 7.4).

### 1.2 Antigen Biotinylation

The purified human CEA N-terminal domain recombinant fragment was site specifically biotinylated using BirA (E. coli biotin ligase) following the protocol of Fairhead et al. (Fairhead et al., 2015). The protein was first dialyzed in BirA buffer (100 mM Tris PH 7.5, 200 mM Nacl, 5 mM MgCl). The biotin-labelling reaction was performed for 24 hours with 1 mg of protein, 28 μL of 40 mM Biotin, 43 μg of BirA, 70 μL of 0.5 M ATP and protease inhibitors. After 24 hours, the protein was purified by size exclusion chromatography and dialyzed back into PBS buffer (pH 7.4).

### 1.3 Antigen Characterization

The recombinantly produced human CEA N-terminal fragment was analyzed by SDS-PAGE and by size exclusion chromatography using a Superdex 75 increase 10/300 GL column on an ÄKTA FPLC.

### 1.4 Isolation of Naïve scFv Antibody Fragments from a Human Phage Display Library

Fully human monoclonal antibodies specific to the N-terminal domain of human CEA were isolated from a scFv phage library, following a previously described protocol (Viti et al., 2000). Briefly, the biotinylated human CEA (final concentration 120 μmol) was immobilized on 60 μL of streptavidin-coated magnetic beads. After blocking in 4% milk-PBS, 800 μL of scFv displaying phage display library was added (1012 transforming units of phage/ml) and the reaction mixture was incubated for 1½ hours. After six washes with 0.1% Tween 20 in PBS and a subsequent six washes with PBS, selected phages were eluted by reducing the disulphide bonds in the biotin linker with triethylamine. Isolated phages were then amplified in E. coli strain TG-1 (using VCS-M13 Interference-Resistant Helper Phage) and precipitated from culture supernatant using 20% polyethylene glycol/2.5 M NaCl and used for a second round of bio-panning.

Following two rounds of bio-panning, individual clones infected with isolated phage were cultured and scFv expression was induced by Isopropyl β-D-1-thiogalactopyranoside (IPTG). Bacterial supernatants containing antibody fragments were collected and screened by ELISA. Based on the ELISA signal, the clone G9 was selected for further characterization.

### 1.5 Construction and Cloning of Affinity Maturation Libraries

The binding kinetics of G9 was improved by two subsequent affinity maturation procedures. Briefly, new phage libraries were cloned by random mutagenesis of residues in the complementary-determining region CDR1 or CDR2 of heavy and light chain (VH and VL).

### 1.5.1 CDR1 Randomization

For the first affinity maturation, primers were designed to randomize of VH and VL of CDR1. Three different fragments were amplified. Fragments 1 and 2 were PCR assembled, and fragments 2 and 3 were also PCR assembled. The resulting two PCR fragments were PCR assembled and the PCR product was digested with NotI and NcoI and ligated into a vector digested with the same enzymes.

### 1.5.2 CDR2 Randomization

For the second affinity maturation primers were designed to randomize VH and VL of CDR2. Three different fragments were amplified. Fragments 1 and 2 were PCR assembled, and fragments 2 and 3 were also PCR assembled. The resulting two PCR fragments were assembled and the PCR product was digested with NotI and NcoI and ligated into a vector digested with same enzymes.

The resulting ligation products were transformed into fresh electrocompetent cells of E. coli strain TG-1 prepared for transformation by washing the cells twice in 1 mM HEPES/5% glycerol and twice with 10% glycerol in water. The cells were then resuspended in 10% glycerol to a density of approximately 2×10¹¹ cells/mL. The cells were subjected to electroporation after mixing with the ligation product, spread on agar plates, and incubated at 30° C. overnight. The following day, cells were collected from the plates and phage were produced by superinfection with helper phage, followed by PEG/NaCl precipitation.

### 1.6 Phage Display Selection on the Affinity Matured CDR1 and CDR2 Libraries of G9

Bio-panning of the affinity maturation library was performed with biotinylated human CEA N-terminal fragment. After one round of panning (as described above), a total of 36 positive clones were identified using ELISA and analyzed for binding to human CEA by BIAcore. The clones with the highest affinity for human CEA “F7” (after the first round of affinity maturation) and “PEA2” (after the second round of affinity maturation) were then further characterized.

### 1.7 Cloning and Expression of G9, F7 and PEA2 scFvs

G9, F7 and PEA2 scFvs were cloned into a vector for protein expression in mammalian cells. The genes for the scFvs were amplified by PCR. The primers were designed to add a signal peptide and restriction sites for HindIII and NotI. The PCR product was digested with HindIII and NotI and ligated into a vector digested with the same enzymes. The clones were expressed by PEI induced transient gene expression (TGE) in CHO-S cells (as described above). The antibody fragments were purified from the cell culture medium by Protein A affinity chromatography and then dialyzed against phosphate buffered saline (PBS, pH7.4). The purified antibody fragments were then analyzed by SDS-PAGE and size exclusion chromatography on a Superdex 75 increase 10/300 GL column on an ÄKTA FPLC. The amino acid sequences of the PEA2 antibody in scFv format is shown in SEQ ID NO: 10. The VH and VL domain sequences (with CDRs underlined) of the G9 antibody are set forth in SEQ ID NOs 42 and 43 respectively. The VH and VL domain sequences (with CDRs underlined) of the F7 antibody are set forth in SEQ ID NOs 44 and 45 respectively.

### 1.8 Affinity Measurement

The affinities of G9, F7 and PEA2 in monomeric scFv format were measured by Surface Plasmon Resonance (SPR) on a BIAcore X100 system. Biotinylated CEA was immobilized on a streptavidin coated sensor chip, yielding a density of 800 RU. Monomeric preparations of scFv fragments were prepared by gel filtration on a Superdex 75 increase 10/300GI column and analyzed in serial 2-fold dilutions at a flow rate of 10 μl/min. The binding curves were analyzed with the BIAevaluation 3.2 software.

### 1.9 Results

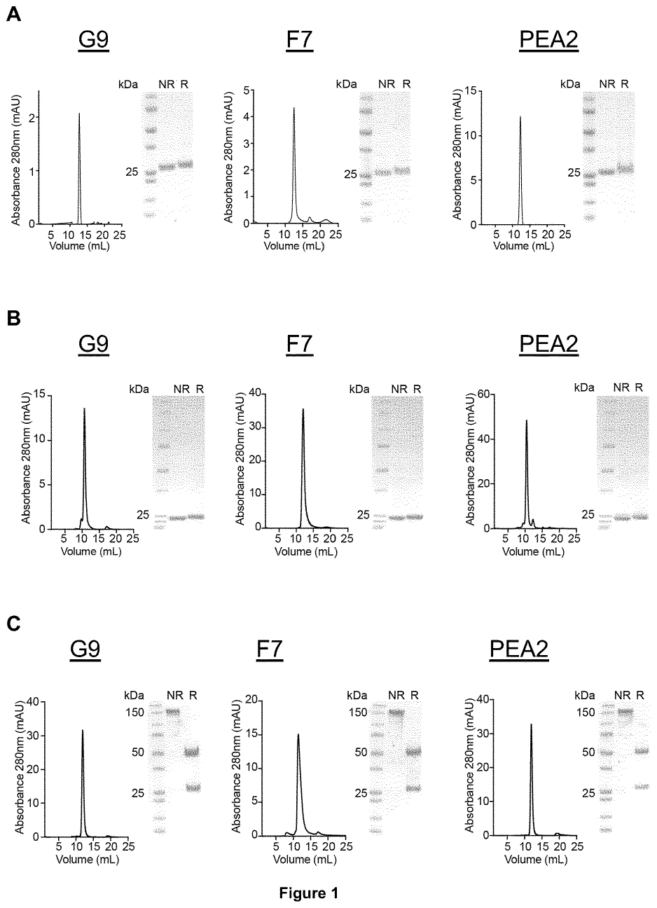

A new anti-CEA fully human antibody termed “G9” was isolated by phage display. To improve affinity, affinity maturation libraries based on G9 were generated. From these libraries the intermediate affinity clone “F7” and the high affinity clone “PEA2” were selected based on their affinity for the N-terminal fragment of human CEA. G9, F7 and PEA2 were characterized by SDS-PAGE and size exclusion chromatography (FIG. 1A). These results showed that each of the G9, F7 and PEA2 scFvs had the expected molecular weights under reducing and non-reducing conditions and also had excellent purity, as evidenced by the single peak observed by SEC for each scFv.

By SPR analysis the affinity of G9, F7 and PEA2 were assessed, resulting in Kd (affinity) values of 640 nM, 50 nM and 7.7 nM, respectively. The Kd values of the affinity matured F7 and PEA2 antibodies each represent a substantial improvement over their respective parent antibodies (FIG. 2A).

### Example 2: Cloning, Expression, and In Vitro Characterization of the G9, F7 and PEA2 Antibodies in IgG1 Format

### 2.1 Cloning of G9, F7 and PEA2 into IgG1 Format

Cloning of the G9, F7 and PEA2 antibodies into the same mammalian cell expression vector as above commenced by cloning the light chain. Primers were designed to add the leader sequence and restriction sites for SpeI and BsiWI. The resulting fragment was digested with SpeI and BsiWI and ligated into a suitable vector previously digested with the same restriction enzymes. The cloning procedure was continued with the cloning of the heavy chain of IgG1.

Primers were designed to add the leader sequence and restriction sites for HindIII and XhoI. The final PCR product was digested with HindIII and XhoI and ligated into the vector mentioned above, already carrying the light chain as insert, previously digested with the same restriction enzymes. The amino acid sequence of the PEA2 antibody in IgG1 format is shown in SEQ ID NOs: 16 and 17. The same cloning strategy was used to prepare the anti-hen egg lysozyme antibody “KSF” in IgG1 format (used herein as a negative control). The amino acid sequence of the KSF antibody in IgG1 format is shown in SEQ ID NOS 38 and 39. The VH and VL domain sequences (with CDRs underlined) of the KSF antibody are set forth in SEQ ID NOs 46 and 47 respectively.

### 2.2 Characterization of G9, F7 and PEA2 in IgG1 Format

The G9, F7 and PEA2 antibodies in IgG1 format were expressed using transient gene expression (TGE) in CHO-S cells and purified by protein A affinity chromatography, dialyzed and stored in PBS (as described above).

Proteins were characterized by SDS-PAGE and size-exclusion chromatography using a Superdex 200 increase 10/300 GL column on an ÄKTA FPLC as described above (FIG. 1C).

### 2.3 Immunofluorescence Analysis

Immunofluorescence experiments were performed with PEA2 and KSF IgG1 (negative control), both in IgG1 format on cryosections of LS174T tumor tissue slides. The PEA2 and KSF IgG1 molecules were conjugated to Fluorescein isothiocyanate (FITC) to allow detection.

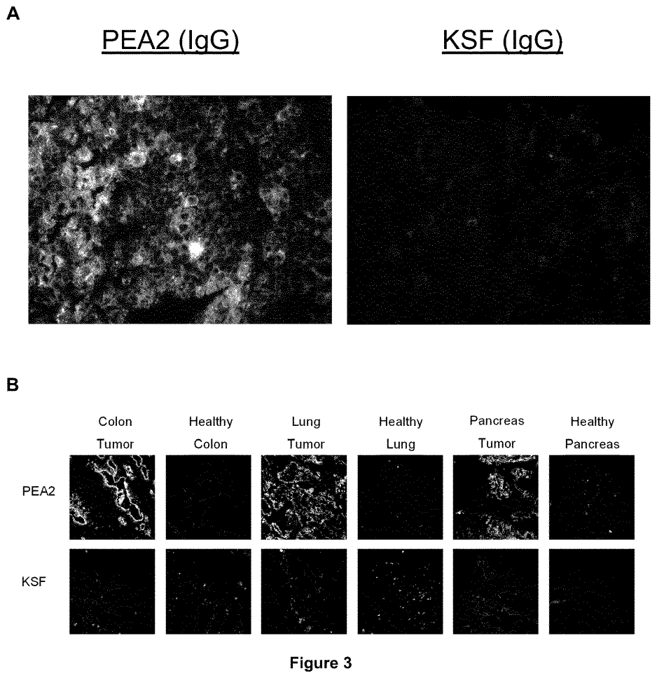

Cryostat sections (10 μm) were stained using the PEA2 and KSF IgG1 conjugates at a final concentration of 10 μg/mL and detected with rabbit anti-FITC antibody (Biorad) and goat-anti-rabbit AlexaFluor488 antibody (Invitrogen). Slides were mounted with fluorescent mounting medium and analyzed with a microscope (FIG. 3A).

Immunofluorescence analysis was also performed on a human tissue microarray (Amsbio, T6235700-5). Prior to staining, samples were fixed in ice-cold acetone. Sections were then stained using FITC-labelled PEA2 and KSF (negative control) antibodies in IgG1 format at a concentration of 50 nM in 2% BSA/PBS and detected using rabbit-anti-FITC antibody (Biorad; 1:1000) and goat-anti-rabbit-Alexa Fluor488 antibody (Invitrogen; 1:500). Tissue vasculature was visualized by staining with mouse anti-human CD31 (Invitrogen; 1:200) and rabbit anti-mouse-Alexa Fluor594 (Invitrogen; 1:500) antibodies. DAPI (Invitrogen) was used to stain cell nuclei. Slides were mounted with fluorescence mounting medium (Dako Agilent) and analyzed with Leika DMI6000B (Leica Microsystems) (FIG. 3B).

### 2.4 Flow Cytometry Analysis

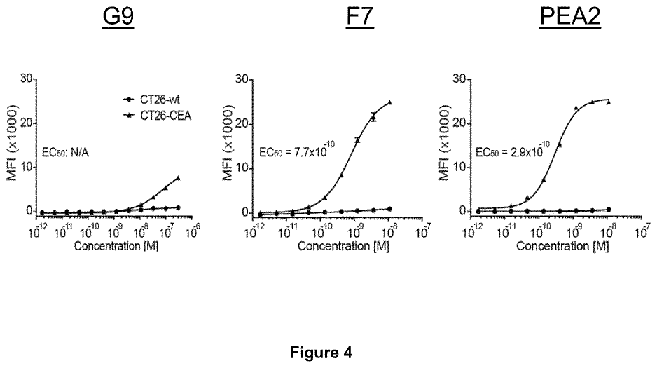

Binding of the G9, F7 and PEA2 antibodies in IgG1 format to CEA on the cell surface was confirmed by flow cytometry on the human CEA transfected murine cell line CT26 at different concentrations of the antibodies with the corresponding wild-type cell line (which does not express human CEA) acting as a negative control. Cells were incubated with the G9, F7 and PEA2 antibodies in IgG1 format and antibody binding was detected through a phycoerythrin (PE) conjugated goat anti-human IgG Fc antibody (Invitrogen). Cells were analysed on a CytoFLEX cytometer (Beckman Coulter) and the raw data processed using the FlowJo 10.4 software (FIG. 4).

### 2.5 Ex-Vivo Experiments

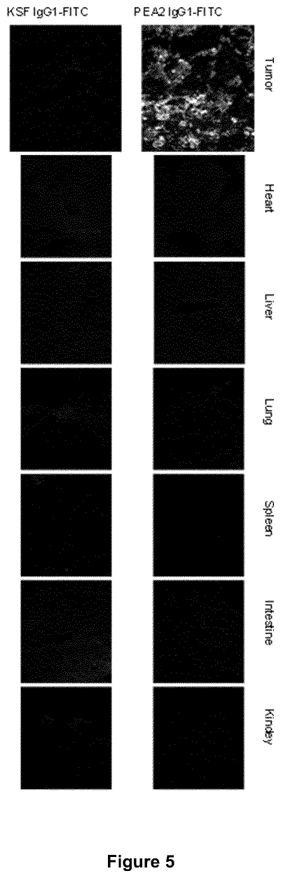

5×10⁶ LS174T human colon adenocarcinoma cells were implanted subcutaneously in the flank of eight-week-old female BALB/c nude mice. For ex-vivo immunofluorescence analysis, when tumors reached a size of 150-250 mm³, mice were injected with 100 μg PEA2 IgG1-FITC and KSF IgG1-FITC and sacrificed 24 hours after injection. Organs were excised and embedded in cryo-embedding medium and cryostat sections (10 μm) were stained using the following antibodies: rabbit anti-FITC and goat anti-rabbit AlexaFluor488. Slides were mounted with fluorescent mounting medium and analyzed using a microscope (FIG. 5).

### 2.6 Results

The G9, F7 and PEA2 antibodies in IgG1 format showed the expected molecular weight under reducing and non-reducing conditions when analysed by SDS-PAGE and good purity as evidenced by the single peak for each antibody observed using SEC (FIG. 1C).

The binding affinity of the G9, F7 and PEA2 antibodies in IgG1 format to human CEA was confirmed by Biacore (FIG. 2C) and flow cytometry (FIG. 4). G9, F7, and PEA2 all showed binding to the CEA positive cell line, while no binding was observed on CEA negative CT26 wild-type cells (FIG. 4). G9 showed moderate binding to CT26-CEA cells in high concentrations. The affinity matured antibodies showed improved binding with functional affinity (KDapp) values of 770 μM and 290 μM for F7 and PEA2, respectively.

Furthermore, the immunofluorescence analysis on LS174T tissue sections using the PEA2 antibody in IgG1 format confirmed that the antibody is capable of binding human CEA in tumor sections (FIG. 3A; left). The KSF antibody in IgG1 format was used as negative control and showed no binding under the same conditions (FIG. 3A; right). The ability of the PEA2 antibody to efficiently target tumors was confirmed through immunofluorescence analysis on a tissue micro array (TMA) containing different human cancer samples and their corresponding healthy controls. PEA2 gave positive results on human lung, pancreatic and colon tumors. No signal was seen upon staining of the healthy corresponding control on the human TMA (FIG. 3B; top). No binding was seen with the negative control KSF (IgG1), as was expected (FIG. 3B; bottom).

The ability of the PEA2 antibody in IgG1 format to efficiently target tumors was also reconfirmed through ex vivo analysis of binding of PEA2 (IgG1) to tumors expressing CEA in human colon adenocarcinoma-bearing mice (FIG. 5; right). No binding was seen with the negative control KSF (IgG1) as was expected (FIG. 5; left).

### Example 3: Cloning, Expression, and Characterization of the G9, F7, and PEA2 Antibodies in Diabody Format

### 3.1 Cloning of G9, F7, and PEA2 into Diabody Format

G9, F7, and PEA2 were cloned in diabody format into a mammalian cell expression vector using the scFv format described above as a template. The primers were designed to shorten the linker between VH and VL to GGSGG (SEQ ID NO: 14), to force homodimerization of two scFv fragments.

Fragments 1 and 2 were assembled by PCR and the resulting fragment was digested with HindIII and NotI and ligated into a suitable vector previously digested with the same restriction enzymes. The amino acid sequence of the PEA2 antibody in diabody format is shown in SEQ ID NO: 12.

The same cloning strategy was used to prepare the anti-hen egg lysozyme antibody “KSF” in diabody format (used herein as a negative control).

### 3.2 Characterization of G9, F7 and PEA2 in Diabody Format

The diabodies were expressed by transient gene expression (TGE) in CHO-S cells and purified by protein A affinity chromatography, dialyzed and stored in PBS (as described above).

Proteins were characterized by SDS-PAGE and size-exclusion chromatography using a Superdex 200 increase 10/300 GL column on an ÄKTA FPLC (FIG. 1B).

### 3.3 Quantitative Biodistribution with Radio-Labelled Diabodies

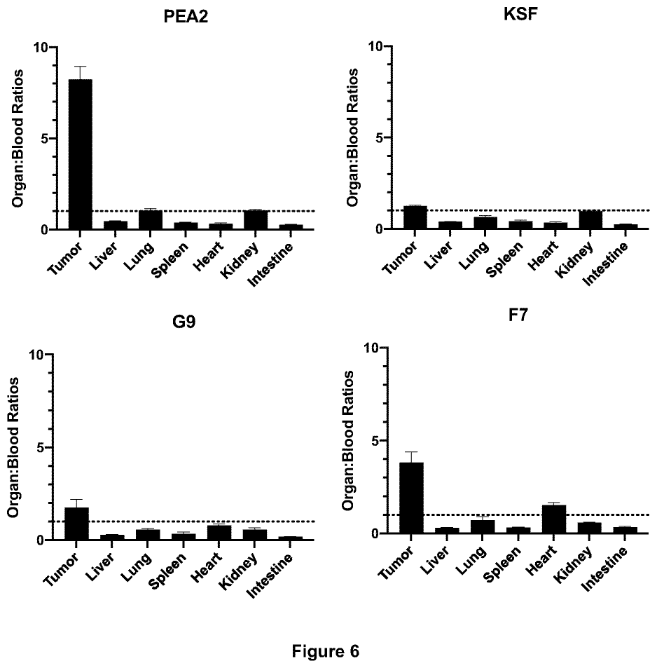

5×10⁶ LS174T human colon adenocarcinoma cells were implanted subcutaneously in the flank of eight-week-old female BALB/c nude mice. G9, F7, PEA2 and KSF (negative control) antibodies in diabody format were radio-iodinated with 125I and chloramine T hydrate and purified on a PD10 column. For quantitative biodistribution analysis, when tumors reached a size of 150-250 mm³, mice were randomized by tumor size (n=4) and the radiolabeled diabodies were injected into the lateral tail vein. Mice were sacrificed 24 h post-injection and their organs were excised, weighed, and measured for radioactivity using a gamma counter (FIG. 6).

### 3.4 Results

The G9, F7 and PEA2 antibodies in diabody format showed the expected molecular weight under reducing and non-reducing conditions when analysed by SDS-PAGE and good purity as evidenced by the single peak for each antibody observed using SEC (FIG. 1B).

The binding affinity of the G9, F7 and PEA2 antibodies in diabody format to human CEA was confirmed by Biacore (FIG. 2B). Furthermore, the ability of the G9, F7 and PEA2 antibodies in diabody format to efficiently target tumors was confirmed through quantitative biodistribution analysis in human colon adenocarcinoma-bearing mice (FIG. 6). Each of the three anti-CEA diabodies had biodistribution profiles confirming selective binding to tumor tissues. In particular, the results of PEA2 confirmed its excellent biodistribution profile of this diabody, as shown by the high organ: blood ratio for the tumor tissue. The biodistribution profile of the PEA2 antibody was notably far superior in terms of tumor accumulation compared with antibodies G9 and F7, demonstrating that the biodistribution profile of antibody PEA2 is not an inherent property of antibodies which bind to the N-terminal fragment of CEA.

### Example 4: Cloning, Expression, and Characterization of the PEA2-mIL12 Conjugate

### 4.1 Cloning Procedure of PEA2-mIL12 Fusion Protein

A PEA2-mIL12 fusion protein was prepared, consisting of the PEA2 antibody in single chain diabody format, fused at its N-terminus to p40 and p35 domains of murine IL-12 connected through a (DIGGGAGGGGAGGGA) linker (SEQ ID NO: 19). The template for the mammalian cell expression vector already carried the genes for p40 and the N terminal part of p35. The gene for the C terminal part of p35 was amplified by PCR and the gene for the first VH and VL domains of PEA2 was amplified by PCR. The two fragments were assembled by PCR, and the resulting fragment was digested with the HindIII and BamHI restriction enzymes and ligated into the vector mentioned above (carrying the p40 and the N-terminal part of the p35 gene) previously digested with the same restriction enzymes.

The gene for the second VH and VL domains of PEA2 was further amplified by PCR and the resulting fragment was digested with the BamHI and NotI restriction enzymes and ligated into the same vector mentioned above, previously digested with the same restriction enzymes. The amino acid sequence of the PEA2-mIL12 fusion protein is shown in SEQ ID NO: 18.

The same cloning strategy was used to produce the KSF-mIL12 conjugate, used herein as a negative control.

### 4.2 Characterization of the PEA2-mIL12 Fusion Protein

The PEA2-mIL12 fusion protein was expressed by transient gene expression (TGE) in CHO-S cells), purified from the cell culture medium by protein A affinity chromatography, dialyzed, and stored in PBS (as described above).

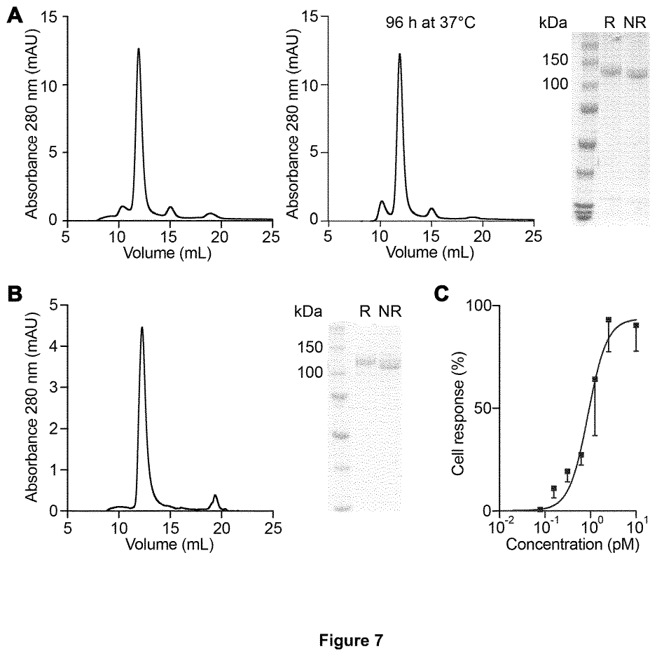

The fusion protein was characterized by SDS-PAGE and size-exclusion chromatography using a Superdex 200 increase 10/300 GL column on an ÄKTA FPLC both after purification, and after incubation at 37° C. for 96 hours (FIG. 7A).

### 4.3 Binding Validation of PEA2-mIL12

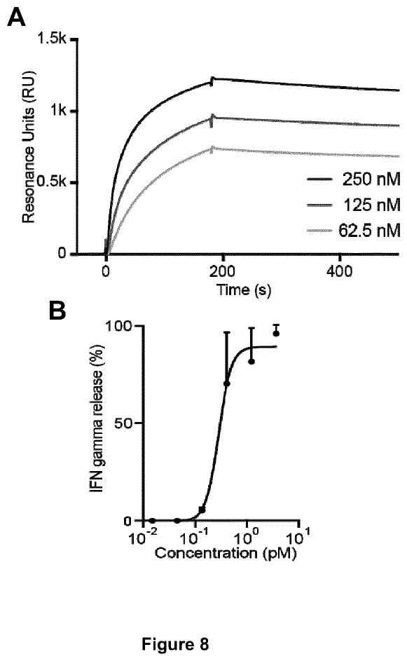

To show that the binding capacity of the PEA2 antibody for the CEA antigen was retained in the conjugate, SPR analysis was performed with the PEA2-mIL12 fusion protein on a BIAcore X100 system. Biotinylated CEA was immobilized on a streptavidin coated sensor chip, yielding a density of 800 RU. Monomeric preparations of the PEA2-mIL12 conjugate were prepared by gel filtration on a Superdex 200 increase 10/300GI column and analyzed in in serial 2-fold dilutions at a flow rate of 10 μl/min. The binding curves were analyzed with the BIAevaluation 3.2 software (FIG. 8A).

### 4.4 Activity Assay of PEA2-mIL12 and KSF-mIL12

To show retained activity of the murine IL12 domain, PEA2-mIL12 and KSF-mIL12 were tested in an IFN-γ release assay. Lymphocytes were isolated from freshly dissected spleens of BALB/c mice. After red blood cell lysis, lymphocytes were resuspended at 3×10⁶ cells/mL in RPMI-1640 supplemented with antibiotic-antimycotic and 10% Fetal Bovine Serum. 100 μL of the cell suspension was incubated for 6 days at 37° C. and 5% CO2 with a serial dilution of the IL12 derivatives. IFN-γ levels from cultured supernatants were analyzed using ELISA (FIGS. 7C and 8B).

### 4.5 Therapy Study with the PEA2-mIL12 Fusion Protein

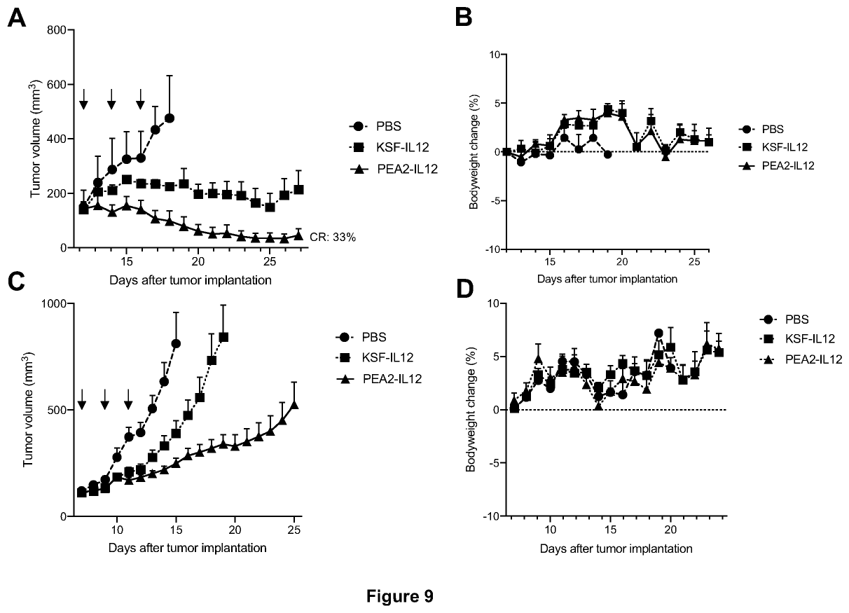

3×10⁶ human CEA-expressing C51 or CT26 colon carcinoma cells were implanted subcutaneously in the flank of eight-week-old female BALB/c mice. Tumor volume was measured with a caliper and volume was calculated using the formula: tumor volume=(Length [mm]*Width² [mm])/2). When tumors reached a suitable volume (approx. 100 mm³), mice were randomized into three groups, and treated using the PEA2-mIL12 and KSF-mIL12 fusion proteins (three i.v. injections of 12 μg every 48 h) and PBS (used as negative control, three i.v. injections every 48 h), respectively. The results are expressed as tumor volume in mm³±SEM. (FIG. 9).

### 4.6 Results

The PEA2 single-chain diabody genetically fused with murine IL-12 and the KSF-mIL12 conjugate both showed the expected molecular weight under reducing and non-reducing conditions when analysed by SDS-PAGE and good purity as evidenced by the single peak observed by SEC (FIGS. 7A and 7B).

The PEA2-mIL12 conjugate showed excellent stability, exhibiting an unchanged SEC profile after a four-day incubation at 37° C. (FIG. 7A). The retained biological activity of the cytokine in the KSF-mIL12 conjugate was confirmed by an IFN-γ release assay (FIG. 7C). The retained biological activity of the binding antibody fragment and the cytokine in the PEA2-mIL12 conjugate were each confirmed by BIAcore analysis (FIG. 8A), and an IFN-γ release assay (FIG. 8B), respectively. Treatment with the PEA2-mIL12 conjugate resulted in a reduction in CT26-CEA tumor volume both in absolute terms, with a complete response in 33% of tested animals, and relative to the negative controls (KSF-mIL12; saline; FIG. 9A). Treatment with the PEA2-mIL12 conjugate resulted in C51-CEA tumor growth retardation compared to the negative controls (KSF-mIL12; saline; FIG. 9C). No notable change in body weight was detected during the therapy in either the CT26 or C51 tumor-bearing mice, indicating good tolerability of the fusion protein at the dose used (FIGS. 9B and 9D). Together, these data confirm both the therapeutic effect and the tolerability of PEA2-mIL12 in mouse tumor models.

### Example 5: Cloning, Expression, and Characterization of the PEA2-IL2 Conjugate

### 5.1 Cloning Procedure of PEA2-IL2 Fusion Protein

A PEA2-IL2 fusion protein was prepared, consisting of the PEA2 antibody in diabody format, fused at its C-terminus to human IL2 connected through a (GGGGS)³ linker (SEQ ID NO: 22). The template for the mammalian cell expression vector already carried the gene for human IL2. The gene for the PEA2 antibody in diabody format was amplified by PCR. The resulting fragment was digested with the HindIII and BamHI restriction enzymes and ligated into the vector mentioned above (carrying the human IL2 gene) previously digested with the same restriction enzymes. The amino acid sequence of the PEA2-IL2 fusion protein is shown in SEQ ID NO: 21.

### 5.2 Characterization of the PEA2-IL2 Fusion Protein

The PEA2-IL2 fusion protein was expressed by transient gene expression (TGE) in CHO-S cells and purified by protein A affinity chromatography, dialyzed, and stored in PBS (as described above).

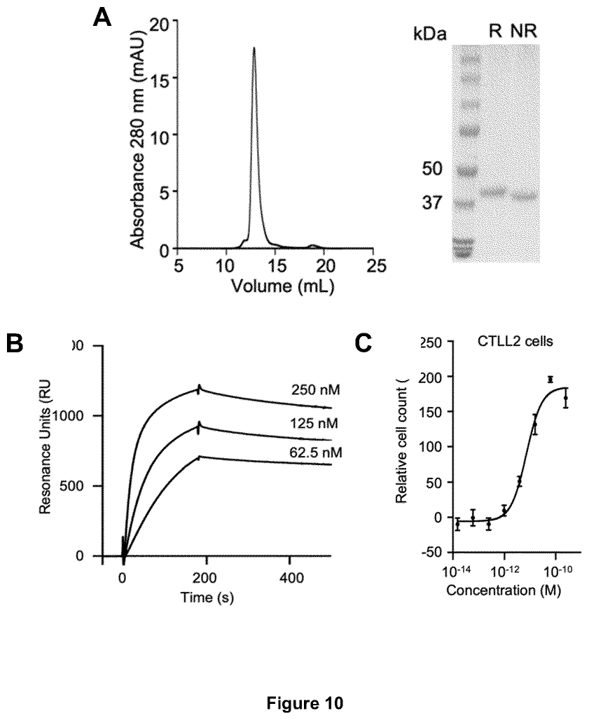

The fusion protein was characterized by SDS-PAGE and size-exclusion chromatography using a Superdex 200 increase 10/300 GL column on an ÄKTA FPLC as described above (FIG. 10A).

### 5.3 Binding Validation of PEA2-IL2

To show that the binding capacity of the PEA2 antibody for the CEA antigen was retained in the conjugate, SPR analysis was performed with the PEA2-IL2 fusion on a BIAcore X100 system. Biotinylated CEA was immobilized on a streptavidin coated sensor chip, yielding a density of 800 RU. Monomeric preparations of the PEA2-IL2 conjugate were prepared by gel filtration on a Superdex 200 increase 10/300GI column and analyzed at serial 2-fold dilutions at a flow rate of 10 μl/min. The binding curves were analyzed with the BIAevaluation 3.2 software (FIG. 10B).

### 5.4 Activity Assay of PEA2-IL2

To show retained activity of the human IL2 domain in the conjugate format, PEA2-IL2 was tested in a cell proliferation assay. CTLL2 cells were grown in RPMI-1640 medium (Gibco; 21875-034), supplemented with 10% FBS and 10% T-STIM with ConA (Corning). Cells were washed and left in starvation medium (RPMI-1640 supplemented with 1% FBS; no T-STIM) for 24 hours. Cells were then incubated for 96 hours in starvation medium supplemented with different concentrations of PEA2-IL2. Cell survival and proliferation was quantified, using the CellTiter 96® AQueous One Solution Cell Proliferation Assay (Promega), following the manufacturer's protocol (FIG. 10C).

### 5.5 Results

The PEA2 diabody genetically fused with human IL2 showed the expected molecular weight under reducing and non-reducing conditions when analysed by SDS-PAGE and good purity as evidenced by the single peak observed by SEC (FIG. 10A). The retained biological activity of the binding antibody fragment and the cytokine were each confirmed by BIAcore analysis (FIG. 10B) and a CTLL2 cell proliferation assay (FIG. 10C), respectively.

### Example 6: Cloning, Expression, and Characterization of the PEA2-mTNF Conjugate

### 6.1 Cloning Procedure of PEA2-mTNF Fusion Protein

A PEA2-mTNF fusion protein was prepared, consisting of the PEA2 antibody in scFv format, fused at its C-terminus to murine TNF connected through a (SSSSG)³ linker (SEQ ID NO: 15). The gene for the PEA2 antibody in scFv format was amplified by PCR. The two fragments were assembled by PCR and the resulting fragment was digested using the restriction enzymes HindIII and NotI and ligated into a suitable vector previously digested with the same restriction enzymes. The amino acid sequence of the PEA2-mTNF fusion protein is shown in SEQ ID NO: 24.

### 6.2 Characterization of the PEA2-mTNF Fusion Protein

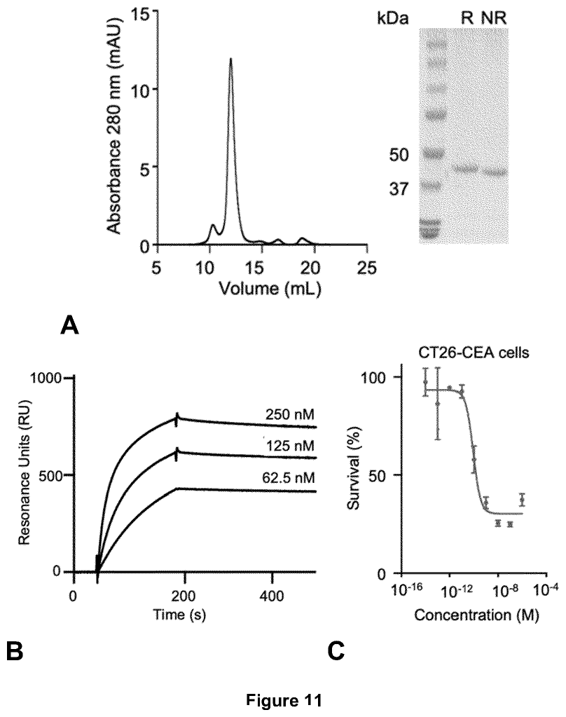

The PEA2-mTNF fusion protein was expressed by transient gene expression (TGE) in CHO-S cells and purified by protein A affinity chromatography, dialyzed, and stored in PBS (as described above). The conjugate was characterized by SDS-PAGE and size-exclusion chromatography using a Superdex 200 increase 10/300 GL column on an ÄKTA FPLC as described previously (FIG. 11A).

### 6.3 Binding Validation of PEA2-mTNF

To show that the binding capacity of the PEA2 antibody for the CEA antigen was retained in the conjugate, SPR analysis was performed with the PEA2-mTNF fusion on a BIAcore X100 system. Biotinylated CEA was immobilized on a streptavidin coated sensor chip, yielding a density of 800 RU. Monomeric preparations of PEA2-mTNF were prepared by gel filtration on a Superdex 200 increase 10/300GI column and analyzed at serial 2-fold dilutions at a flow rate of 10 μl/min. The binding curves were analyzed with the BIAevaluation 3.2 software (FIG. 11B).

### 6.4 Activity Assay of PEA2-mTNF

To show retained activity of the murine TNF domain in the conjugate format, PEA2-mTNF was tested in a cell killing assay. CT26-CEA cells were cultured in DMEM, supplemented with 10% FBS and 2 μg/mL actinomycin D (SBR00013-ML, Sigma), at a density of 250,000 cells/mL. Cells were incubated with different concentrations of PEA2-mTNF for 48 hours. Cell viability was then quantified, using the CellTiter 96® AQueous One Solution Cell Proliferation Assay (Promega), following the manufacturer's protocol. (FIG. 11C).

### 6.5 Results

The PEA2 diabody genetically fused with murine TNF showed the expected molecular weight under reducing and non-reducing conditions when analysed by SDS-PAGE and good purity as evidenced by the single peak observed by SEC (FIG. 11A). The retained biological activity of the binding antibody fragment and the cytokine were each confirmed by BIAcore analysis (FIG. 11B) and a CT26-CEA cell killing assay (FIG. 11C), respectively.

### Example 7: Cloning, Expression, and Characterization of the PEA2-Anti-CD3 (PUB4) Bispecific Antibody in Bispecific T Cell Engager Format

### 7.1 Expression and Characterization of PEA2-PUB4 Bispecific Antibody in Bispecific T Cell Engager Format

The gene of the PEA2-PUB4 bispecific antibody was inserted in the mammalian cell expression vector pcDNA3.1 (+) and was expressed by transient gene expression (TGE) in CHO-S cells and purified by protein A affinity chromatography, dialyzed, and stored in PBS. Proteins were characterized by SDS-PAGE and size-exclusion chromatography using a Superdex 200 increase 10/300 GL column on an ÄKTA FPLC.

The sequence of the PEA2-anti-CD3 (PUB4) bispecific antibody in bispecific T cell engager format is shown in SEQ ID NO: 33.

The humanised anti-CD3 antibody (PUB4) is described in Liu Y, et al. (2022).

### 7.2 Binding Validation of PEA2-PUB4 Bispecific Antibody in Bispecific T Cell Engager Format

Binding of the PEA2-PUB4 bispecific antibody to CEA-positive tumor cells (LS174T) and human T cells was shown by flow cytometry. LS174T cells were detached from cell culture plate using Accutase cell detachment solution. Peripheral blood mononucleated cells (PBMCs) were isolated from blood of healthy donors by density centrifugation using Ficoll-Paque plus. Primary T cells were separated by negative selection using EasySep™Human T cell isolation kit according to the manufacturer's instructions (STEMCELL™ Technologies). Cells were blocked for unspecific binding and incubated with PEA2-PUB4 bispecific antibody. Antibody binding was detected through a 6× Histidine tag, using a PE-conjugated secondary antibody. All staining and washing steps were carried out in cold FACS buffer (2% FBS, 2 mM EDTA in PBS; pH 7.4). Data was acquired on a CytoFLEX cytometer (Beckman Coulter) and processed with the FlowJo 10.4 software.

### 7.3 Activity Assay of PEA2-PUB4 Bispecific Antibody in Bispecific T Cell Engager Format

The biological activity of this bispecific antibody was shown in a LS174T cell killing assay. Target cells (LS174T) were cultured in 96 well plates (20′000 cells/well). After two hours, human T cells (100′000 cells/well) and the PEA2-PUB4 bispecific antibody in different concentrations were added to each well. A condition of no added antibody was also included as a negative control. After 48 hours incubation, the cells were detached using Accutase cell detachment solution. Cells were washed with PBS before staining with zombie violet live/dead staining and incubated for 30 minutes at 4° C. in the dark. Cells were washed once with FACS buffer and stained with an antibody master mix containing anti-mouse CD3-APC and anti CEA-FITC secondary antibody. Data was acquired on a CytoFLEX cytometer (Beckman Coulter) and processed with the FlowJo 10.4 software, to quantify target cell killing.

### 7.4 Results

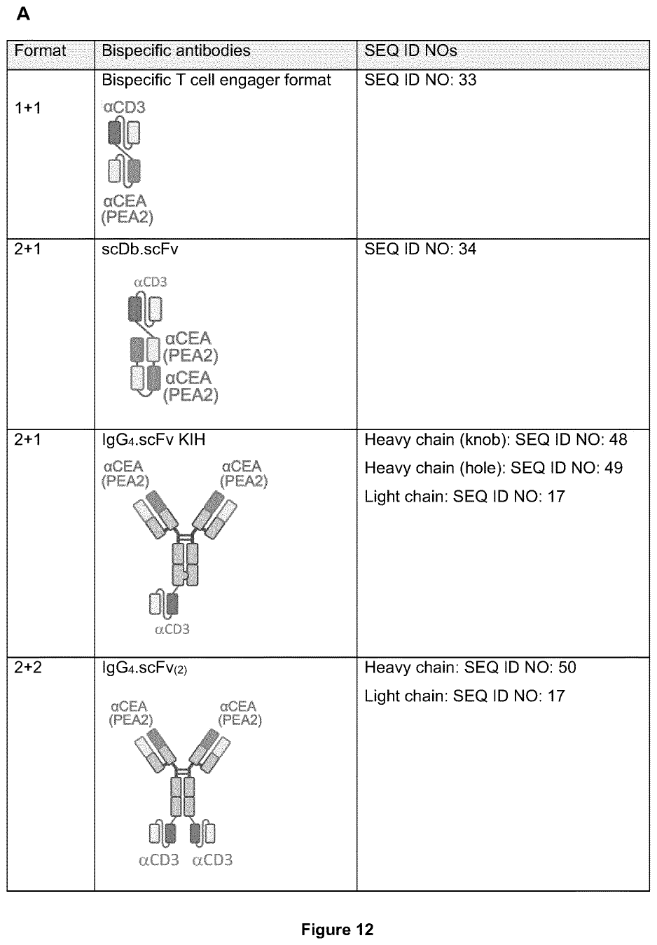

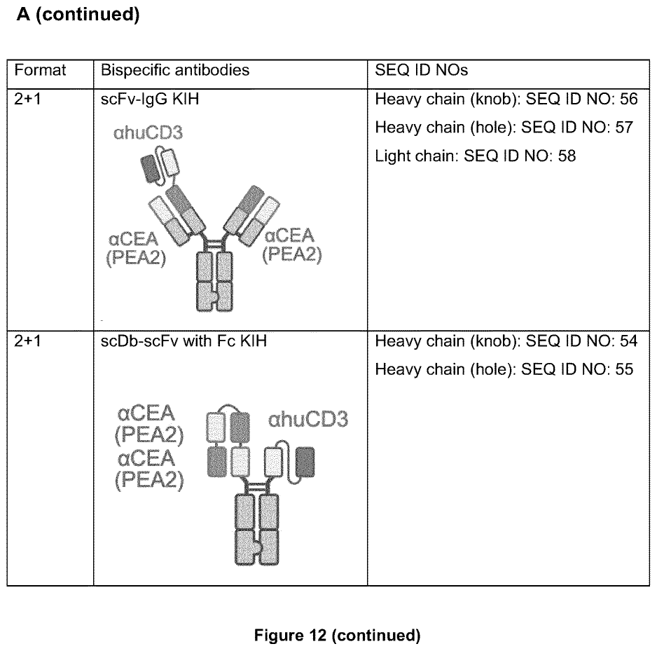

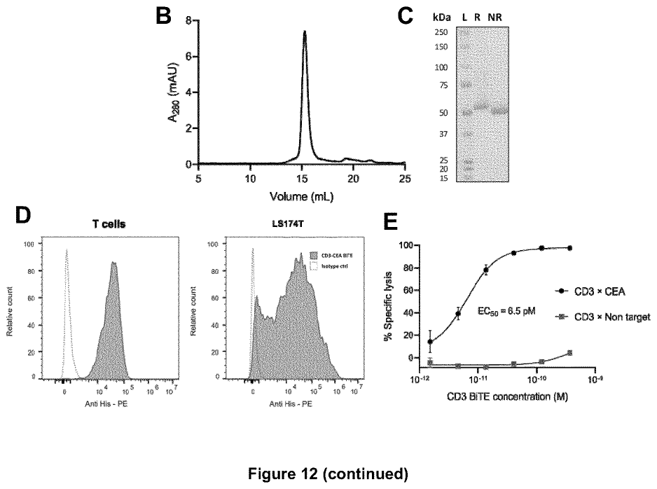

The PEA2-PUB4 bispecific antibody in bispecific T cell engager format (shown in the first row of FIG. 12A) showed the expected molecular weight under reducing and non-reducing conditions when analysed by SDS-PAGE and good purity as evidenced by the single peak observed by SEC (FIG. 12B and FIG. 12C). Binding of the bispecific antibody to T cells and CEA-positive tumor cells was confirmed by flow cytometry (FIG. 12D). The biological activity of this PEA2-PUB4 bispecific antibody was shown in a LS174T cell killing assay (FIG. 12E).

### Example 8: Flow Cytometry Analysis of the PEA2 Antibody on CHO Cells Transiently Expressing CEA, CEACAM1, or CEACAM6 Antigens

### 8.1 Material and Methods

To test the PEA2 antibody for cross-reactivity towards the N-terminal domains of CEACAM1 and CEACAM6, which are highly conserved relative to the N-terminal domain of CEA (CEA=CEACAM5), CHO cells were transiently transfected with the corresponding expression vectors. Flow cytometry was performed 24 h after transfection. Briefly, cells were stained with the antibody PEA2 or a positive control antibody which is cross-reactive with the N-terminal domains of CEA (CEACAM5), CEACAM1, and CEACAM6 (BioLegend; clone 5B2) in murine IgG2a format (50 nM). For detection, a PE-conjugated goat anti-mouse IgG antibody (Invitrogen; 12-4010-82) was used.

All staining and washing steps were carried out in cold FACS buffer (2% FBS, 2 mM EDTA in PBS; pH 7.4). Data was acquired on a CytoFLEX cytometer (Beckman Coulter) and the CytExpert software and processed with the FlowJo 10.4 software.

### 8.2 Results

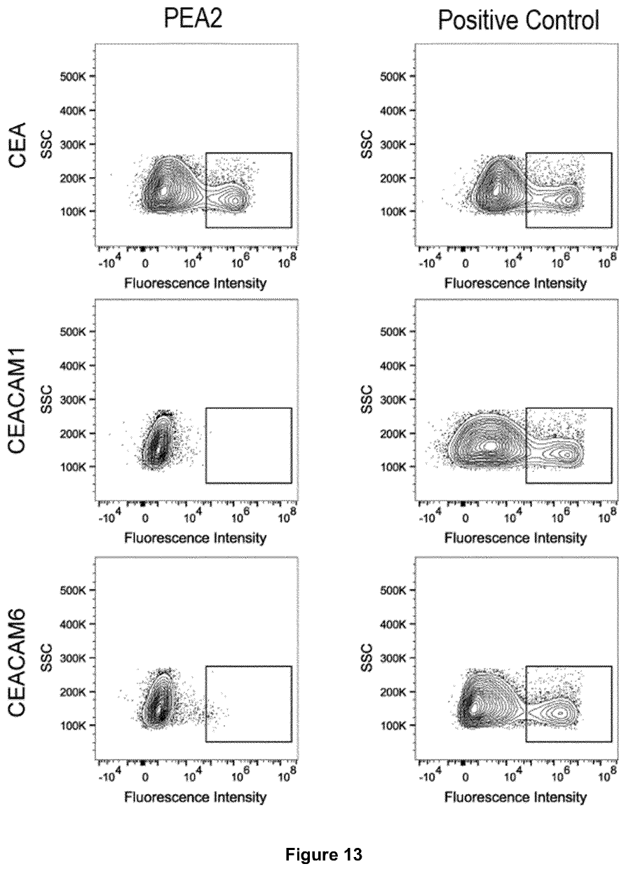

The PEA2 and 5B2 antibodies both showed binding to the CEA-positive cell line, whereas only the positive control antibody 5B2 showed binding to the CEACAM1- and CEACAM6-expressing cell lines. No cross-reactivity was observed for the PEA2 antibody towards CEACAM1 or CEACAM6, confirming the specificity of the PEA2 antibody for CEA (CEACAM5) (FIG. 13).

### Example 9: Cloning, Expression, and Characterization of the Anti-CEA (PEA2)-Anti-CD3 (PUB4) Bispecific Antibody in scDb-scFv with Fc KIH and scFv-IgG KIH Formats

### 9.1 Expression and Characterization of PEA2-PUB4 Bispecific Antibody in scDb-scFv with Fc KIH Format And in scFv-IgG KIH Format

The genes of the PEA2-PUB4 bispecific antibodies were inserted into the mammalian cell expression vector pMM137 and were expressed by PEI-induced transient gene expression in CHO cells, followed by purification to homogeneity by protein A affinity chromatography. Quality control of the produced TCBs was performed by size-exclusion chromatography (SEC; Superdex 200 10/300GL, GE Healthcare).

The sequence of the PEA2-anti-CD3 (PUB4) bispecific antibody in scDb-scFv with Fc KIH format is shown in SEQ ID NOs: 54 and 55 (FIG. 12A). The sequence of the PEA2-anti-CD3 (PUB4) bispecific antibody in scFv-IgG4 KIH format is shown in SEQ ID NOs: 56, 57 and 58 (FIG. 12A). The humanised anti-CD3 antibody (PUB4) is described in Liu Y, et al. (2022).9.2 Binding validation of PEA2-PUB4 bispecific antibody in scDb-scFv with Fc KIH and scFv-IgG KIH formats.

### Cell Lines

PBMCs were extracted from healthy blood donors by density centrifugation using Ficoll-Paque plus. PBMCs were cryopreserved in freezing media composed of 90% FBS and 10% DMSO, and subsequently stored in liquid nitrogen until use. For cell culture, T cell media were prepared using Advanced RPMI 1640 supplemented with 10% FBS, 1% Penicillin-Streptomycin, and 1% GlutaMAX™. CHO cells and LS174T and expanded according to the manufacturer's protocol.

### Flow Cytometry Experiments

Flow cytometry was performed using 50 nM of the bispecific antibodies on both target cells (CEA-expressing MC38) and effector cells (human T cells). Cells (100′000 cells per well) were blocked with FACS buffer (PBS, 2% BSA, and 2 mM EDTA) for 30 minutes. The bispecific antibodies were added to cells and incubated for 30 minutes at 4° C. Following two washing steps with FACS buffer, the primary stained cells were incubated with goat anti-human IgG Fc secondary antibody conjugated to PE for 30 minutes at 4° C. in the dark. The secondary antibody was also used as a negative control. After two washes with PBS, cells were incubated with Zombie Violet live/dead staining for 30 minutes at 4° C. in the dark. Data were acquired on a Cytoflex S flow cytometer (Beckman Coulter) and analyzed using the FlowJo software v10 (BD Biosciences).

### 9.3 Results

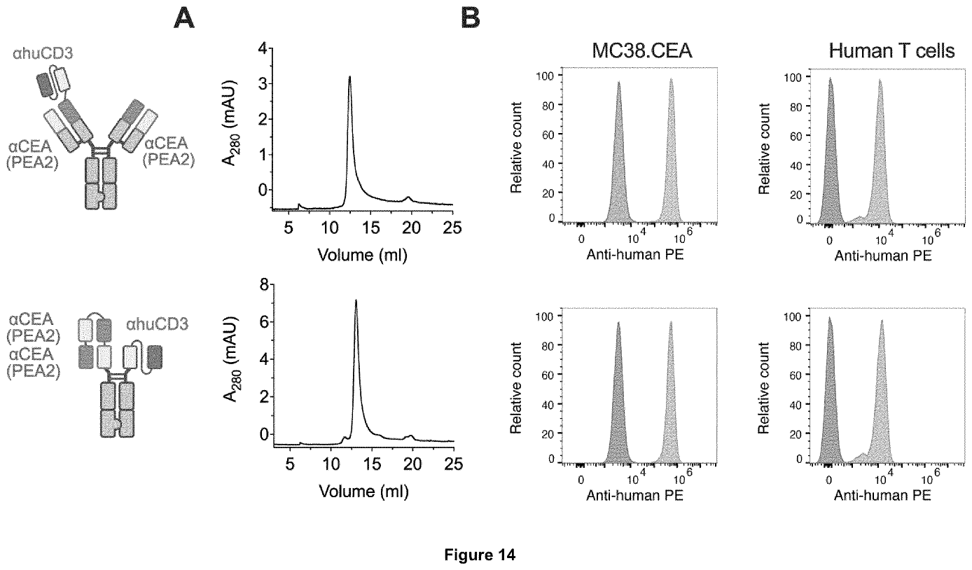

The PEA2-PUB4 bispecific antibodies in scDb-scFv with Fc KIH and scFv-IgG4 KIH formats (shown in FIG. 12A) showed good purity as evidenced by the single peak observed by SEC (FIG. 14A). Binding of the bispecific antibodies to T cells and CEA-positive tumor cells was confirmed by flow cytometry (FIG. 14B).

### Example 10: Cloning, Expression, and Characterization of the Anti-CD28-Anti-CEA Bispecific Antibodies in (2+2) IgG-(scFV)₂ Format

### 10.1 Expression and Characterization of Anti-CD28 (AE2P)-Anti-CEA (PEA2) Bispecific Antibody in (2+2) IgG4-(scFv)₂ Format

The gene of the anti-CD28 (AE2P)-anti-CEA (PEA2) bispecific antibody was inserted into the mammalian cell expression vector pMM137 and was expressed by PEI-induced transient gene expression in CHO cells, followed by purification to homogeneity by protein A affinity chromatography. Quality control of the produced TCBs was performed by size-exclusion chromatography (SEC; Superdex 200 10/300GL, GE Healthcare).

The human anti-CD28 antibody (AE2P) is described in PCT/EP2023/070320.

The sequence of the anti-CD28 (AE2P)-anti-CEA (PEA2) bispecific antibody in (2+2) IgG4-(scFv)₂ format is shown in SEQ ID NOs: 59 and 60 (FIG. 15C).

### 10.2 Expression and Characterization of Anti-CD28 (AE2P)-Anti-CEA (Sm3E) Bispecific Antibody in (2+2) IgG4-(scFv)₂ Format

The gene of the anti-CD28 (AE2P)-anti-CEA (Sm3E) bispecific antibody was inserted into the mammalian cell expression vector pMM137 and was expressed by PEI-induced transient gene expression in CHO cells, followed by purification to homogeneity by protein A affinity chromatography. Quality control of the produced TCBs was performed by size-exclusion chromatography (SEC; Superdex 200 10/300GL, GE Healthcare).

The human anti-CD28 antibody (AE2P) is described in PCT/EP2023/070320.

The sequence of the anti-CD28 (AE2P)-anti-CEA (Sm3E) bispecific antibody in (2+2) IgG4-(scFv)₂ format is shown in SEQ ID NOs: 61 and 62 (FIG. 15C).

### 10.3 Binding Validation of Anti-CD28-Anti-CEA Bispecific Antibodies in (2+2) IgG4-(scFv)₂ Format

The cell lines are described above in section 9.2.

Flow cytometry was performed using 50 nM of the bispecific antibodies on both target cells (CEA-expressing MC38) and effector cells (human T cells). Cells (100′000 cells per well) were blocked with FACS buffer (PBS, 2% BSA, and 2 mM EDTA) for 30 minutes. The bispecific antibodies were added to cells and incubated for 30 minutes at 4° C. Following two washing steps with FACS buffer, the primary stained cells were incubated with goat anti-human IgG Fc secondary antibody conjugated to PE for 30 minutes at 4° C. in the dark. The secondary antibody was also used as a negative control. After two washes with PBS, cells were incubated with Zombie Violet live/dead staining for 30 minutes at 4° C. in the dark. Data were acquired on a Cytoflex S flow cytometer (Beckman Coulter) and analyzed using the FlowJo software v10 (BD Biosciences).

### 10.4 Results

Two different CD28-targeting bispecific antibodies using the fully human anti-CD28 AE2P antibody were produced (FIG. 15C). Both bispecific antibodies were configured in a (2+2) format and also targeted CEA [IgG_{CD28}-(scFv_{CEA})₂]. They differed in their epitope specificity: one bispecific antibody bound to the same epitope as the CD3-targeting bispecific antibodies of Example 9 using the human anti-CEA PEA2 antibody of the invention (IgG_{4 AE2P}-(scFv_{PEA2})₂), while the other bound to a different epitope using the humanized anti-CEA Sm3E antibody (IgG_{4 AE2P}-(scFv_{Sm3E})₂). Both antibodies showed good purity, as evidenced by the single peak observed by SEC (FIG. 15A). Their binding to target cells (CEA-expressing MC38) and effector cells (T cells) was validated by flow cytometry, as shown in FIG. 15B.

### Example 11: In Vitro Activity Assay Using Anti-CD3-Anti-CEA Bispecific Antibodies and Anti-CD28-Anti-CEA Bispecific Antibodies

### 11.1 In Vitro Killing Assay

LS174T cells expressing CEA were seeded in a 96-well plate at a density of 20,000 cells per well. Freshly thawed human PBMCs were added at a 2:1 effector-to-target ratio. Different concentrations of CD3-targeting bispecific antibodies (two different formats: scDb-scFv with Fc KIH and scFv-IgG KIH) were added alone in a serial dilution.

In one experimental setup (same epitope strategy), the anti-CD28-anti-CEA bispecific antibody comprising the PEA2 antibody of the invention was added in equimolar concentrations to each of the CD3-targeting bispecific antibodies.

In another experimental setup (different epitope strategy), the anti-CD28-anti-CEA bispecific antibody comprising the Sm3E antibody was added at a fixed concentration of 10 nM to each of the CD3-targeting bispecific antibodies.

Control conditions were included such as cells only (PBMCs with LS174T cells) and anti-CD28-anti-CEA bispecific antibody only. After a 5-day incubation, cells were detached using an Accutase® Cell detachment solution. The remaining pellet and debris were collected, washed with PBS, and stained with Zombie Violet live/dead staining for 30 minutes at 4° C. in the dark. Following one wash with FACS buffer, cells were stained with an antibody master mix containing anti-human CD3 APC and anti-CEA FITC. Target and effector cells were discriminated based on CEA expression, and the percentage of dead cells was calculated by gating. Additionally, the absolute count of live CD3+ T cells was calculated to evaluate the T cell proliferation.

### 11.2 Results

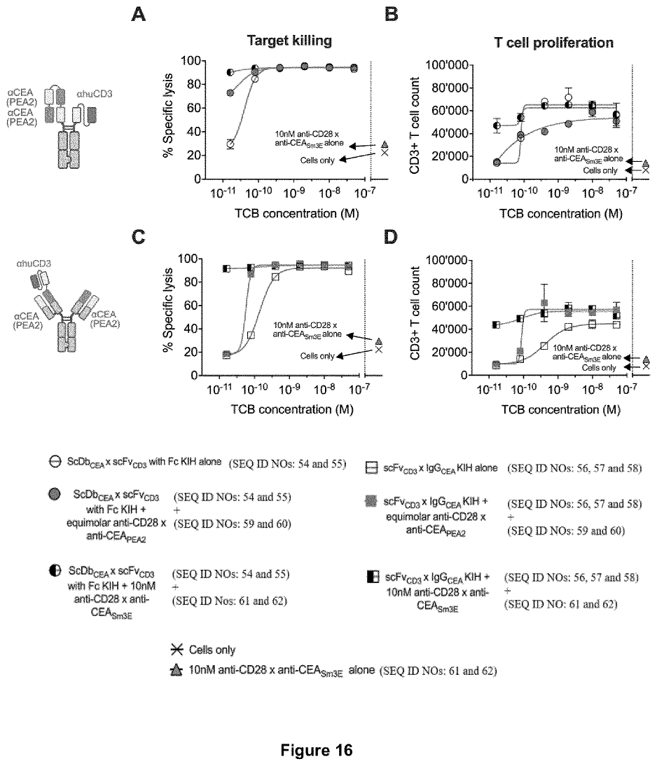

CD3-targeting bispecific antibodies can induce an anti-tumor activity by bridging T cells to cancer cells. However, the absence of co-stimulation can limit T cell activity, leading to early exhaustion and only moderate T cell proliferation. The activity of combinations comprising an anti-CD3-anti-CEA bispecific antibody and an anti-CD28-anti-CEA bispecific antibody was therefore investigated in an in vitro killing assay. Human PBMCs and LS174T cells (naturally expressing CEA) were incubated in a 2:1 effector-to-target ratio for 5 days. Different concentrations of anti-CD3-anti-CEA bispecific antibodies were added in the presence or absence of anti-CD28-anti-CEA bispecific antibodies. Two different anti-CD3 (PUB4)-anti-CEA (PEA2) bispecific antibodies_formats were used: scDb_{PEA2}-scFv_{PUB4} with Fc KIH (FIG. 16, diagram at upper left) and scFv_{Pub4}-IgG_{4 PEA2} KIH (FIG. 16, diagram at lower left). Two distinct anti-CD28-anti-CEA bispecific antibodies (IgG _{CD28}-(scFv_{CEA})₂) were added to the anti-CD3-anti-CEA bispecific antibodies: IgG_{4 AE2P}-(scF_{VPEA2})₂ bispecific antibody (for same epitope strategy using equimolar concentrations) or the IgG4 AE2P-(scFv_{Sm3E})₂ (for different epitope strategy using 10 nM fixed concentration). As a negative control, PBMCs and LS174T cells only were used.

In terms of target cell killing, both formats of anti-CD3-anti-CEA bispecific antibodies exhibited potent cell killing activity in the picomolar range. The scDb_{CEA}-scFv_{CD3} with Fc KIH format was 3.5-fold more potent than the scFv_{CD3}-IgG_{CEA} KIH format, with EC₅₀ values of 40 PM and 140 μM, respectively (FIGS. 16A and 16C). The cell killing activity was significantly enhanced when the anti-CD3-anti-CEA bispecific antibodies were combined with anti-CD28-anti-CEA bispecific antibodies, showing a synergic activity (FIGS. 16A and 16C). Specifically, the combination in the different epitope strategy demonstrated superior potency, with the EC₅₀ value remaining unreached (FIGS. 16A and 16C). T cell proliferation was observed, mirroring the trend seen in the target cell killing assay (FIGS. 16B and 16D). The anti-CD28-anti-CEA bispecific antibody alone did not result in noticeable specific killing or T cell proliferation, highlighting that the synergism is specific and occurs only in the presence of the signal from the anti-CD3-anti-CEA bispecific antibodies.

Sequence Listing

SEQ ID NO: 1-Amino acid sequence of the N-terminal domain of the human carcinoembryonic antigen
(CEACAM5):
KLTIESTPFNVAEGKEVLLLVHNLPQHLFGYSWYKGERVDGNRQIIGYVIGTQQATPGPAYSGREIIYPNA
SLLIQNIIQNDTGFYTLHVIKSDLVNEEATGQFRVYPELPKPSIS
SEQ ID NO: 2-Amino acid sequence of PEA2 CDR1 VH
GFTFSRTAMS
SEQ ID NO: 3-Amino acid sequence of PEA2 CDR2 VH
AIDYDGGVTYYADSVKG
SEQ ID NO: 4-Amino acid sequence of PEA2 CDR3 VH
LTYARFDY
SEQ ID NO: 5-Amino acid sequence of PEA2 CDR1 VL
RASQSVSQNHLA
SEQ ID NO: 6-Amino acid sequence of PEA2 CDR2 VL
LASRRHT
SEQ ID NO: 7-Amino acid sequence of PEA2 CDR3 VL
QQSGRVPWT
SEQ ID NO: 8-Amino acid sequence of the PEA2 VH domain
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSS
SEQ ID NO: 9-Amino acid sequence of the PEA2 VL domain
EIVLTQSPGTLSLSPGERATLSCRASQSVSQNHLAWYQQKPGQAPRLLIYLASRRHTGIPDRFSGSGSGT
DFTLTISRLEPEDFAVYYCQQSGRVPWTFGQGTKVEIK
SEQ ID NO: 10-Amino acid sequence of the PEA2 antibody in scFv format
The linker sequence is underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGGGSGGGGSGGGGEIVLT
QSPGTLSLSPGERATLSCRASQSVSQNHLAWYQQKPGQAPRLLIYLASRRHTGIPDRFSGSGSGTDFTL
TISRLEPEDFAVYYCQQSGRVPWTFGQGTKVEIK
SEQ ID NO: 11-Amino acid sequence of the linker between VH and VL in the scFv fragments
GGGGSGGGGSGGGG
SEQ ID NO: 12-Amino acid sequence of the PEA2 antibody in diabody format (Db)
The linker sequence is underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGSGGEIVLTQSPGTLSLSPG
ERATLSCRASQSVSQNHLAWYQQKPGQAPRLLIYLASRRHTGIPDRFSGSGSGTDFTLTISRLEPEDFAV
YYCQQSGRVPWTFGQGTKVEIK
SEQ ID NO: 13-Amino acid sequence of the PEA2 antibody in single-chain diabody format (scDb)
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGSGGEIVLTQSPGTLSLSPG
ERATLSCRASQSVSQNHLAWYQQKPGQAPRLLIYLASRRHTGIPDRFSGSGSGTDFTLTISRLEPEDFAV
YYCQQSGRVPWTFGQGTKVEIKSSSSGSSSSGSSSSGEVQLLESGGGLVQPGGSLRLSCAASGFTFSR
TAMSWVRQAPGKGLEWVSAIDYDGGVTYYADSVKGRFTISRDNSKNTLYLQMNSLRAEDTAVYYCAKLT
YARFDYWGQGTLVTVSSGGSGGEIVLTQSPGTLSLSPGERATLSCRASQSVSQNHLAWYQQKPGQAPR
LLIYLASRRHTGIPDRFSGSGSGTDFTLTISRLEPEDFAVYYCQQSGRVPWTFGQGTKVEIK
SEQ ID NO: 14-Amino acid sequence of the linker between VH and VL in the diabody (Db) fragments
GGSGG
SEQ ID NO: 15-Amino acid sequence of the flexible linker connecting the two sets of VH and VL
domains in the PEA2 scDb, connecting the PEA2 scFv and mTNF in the PEA2-mTNF fusion protein,
connecting the PEA2 scFv and hTNF in the PEA2-hTNF fusion protein and connecting PEA2 scFv and
hTNFα^{mut} in the IL2-PEA2-TNFα^{mut} conjugate
SSSSGSSSSGSSSSG
SEQ ID NO: 16-Amino acid sequence of the PEA2 heavy chain in IgG1 format
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 17-Amino acid sequence of the PEA2 light chain in IgG (e.g. IgG1 or IgG4) format and in
the PEA2-PUB4 IgG4-scFv (2 + 1) bispecific antibody and PEA2-PUB4 IgG4-(scFv)₂ (2 + 2) bispecific
antibody
EIVLTQSPGTLSLSPGERATLSCRASQSVSQNHLAWYQQKPGQAPRLLIYLASRRHTGIPDRFSGSGSGT
DFTLTISRLEPEDFAVYYCQQSGRVPWTFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC
SEQ ID NO: 18-Amino acid sequence of the PEA2-mIL12 conjugate
The linker sequences are underlined.
MWELEKDVYVVEVDWTPDAPGETVNLTCDTPEEDDITWTSDQRHGVIGSGKTLTITVKEFLDAGQYTCH
KGGETLSHSHLLLHKKENGIWSTEILKNFKNKTFLKCEAPNYSGRFTCSWLVQRNMDLKFNIKSSSSSPD
SRAVTCGMASLSAEKVTLDQRDYEKYSVSCQEDVTCPTAEETLPIELALEARQQNKYENYSTSFFIRDIIK
PDPPKNLQMRPLKNSQVEVSWEYPDSWSTPHSYFSLKFFVRIQRKKEKMKETEEGCNQKGAFLVERTS
TEVQCKGGNVCVQAQDRYYNSSCSKWACVPCRVRSGGGGSGGGGSGGGGSRVIPVSGPARCLSQSR
NLLKTTDDMVKTAREKLKHYSCTAEDIDHEDITRDQTSTLKTCLPLELHKNESCLATRETSSTTRGSCLPP
QKTSLMMTLCLGSIYEDLKMYQTEFQAINAALQNHNHQQIILDKGMLVAIDELMQSLNHNGETLRQKPPV
GEADPYRVKMKLCILLHAFSTRVVTINRVMGYLSSADIGGGAGGGGAGGGAEVQLLESGGGLVQPGGSL
RLSCAASGFTFSRTAMSWVRQAPGKGLEWVSAIDYDGGVTYYADSVKGRFTISRDNSKNTLYLQMNSL
RAEDTAVYYCAKLTYARFDYWGQGTLVTVSSGGSGGEIVLTQSPGTLSLSPGERATLSCRASQSVSQNH
LAWYQQKPGQAPRLLIYLASRRHTGIPDRFSGSGSGTDFTLTISRLEPEDFAVYYCQQSGRVPWTFGQG
TKVEIKSSSSGSSSSGSSSSGEVQLLESGGGLVQPGGSLRLSCAASGFTFSRTAMSWVRQAPGKGLEW
VSAIDYDGGVTYYADSVKGRFTISRDNSKNTLYLQMNSLRAEDTAVYYCAKLTYARFDYWGQGTLVTVS
SGGSGGEIVLTQSPGTLSLSPGERATLSCRASQSVSQNHLAWYQQKPGQAPRLLIYLASRRHTGIPDRF
SGSGSGTDFTLTISRLEPEDFAVYYCQQSGRVPWTFGQGTKVEIK
SEQ ID NO: 19-Amino acid sequence of the flexible linker connecting the mIL12 and the PEA2 scDb in
the PEA2-mIL12 conjugate
DIGGGAGGGGAGGGA
SEQ ID NO: 20-Amino acid sequence of murine IL12 (mIL12)
The linker sequence is underlined
MWELEKDVYVVEVDWTPDAPGETVNLTCDTPEEDDITWTSDQRHGVIGSGKTLTITVKEFLDAGQYTCH
KGGETLSHSHLLLHKKENGIWSTEILKNFKNKTFLKCEAPNYSGRFTCSWLVQRNMDLKFNIKSSSSSPD
SRAVTCGMASLSAEKVTLDQRDYEKYSVSCQEDVTCPTAEETLPIELALEARQQNKYENYSTSFFIRDIIK
PDPPKNLQMRPLKNSQVEVSWEYPDSWSTPHSYFSLKFFVRIQRKKEKMKETEEGCNQKGAFLVERTS
TEVQCKGGNVCVQAQDRYYNSSCSKWACVPCRVRSGGGGSGGGGSGGGGSRVIPVSGPARCLSQSR
NLLKTTDDMVKTAREKLKHYSCTAEDIDHEDITRDQTSTLKTCLPLELHKNESCLATRETSSTTRGSCLPP
QKTSLMMTLCLGSIYEDLKMYQTEFQAINAALQNHNHQQIILDKGMLVAIDELMQSLNHNGETLRQKPPV
GEADPYRVKMKLCILLHAFSTRVVTINRVMGYLSSA
SEQ ID NO: 21-Amino acid sequence of the PEA2-hIL2 conjugate
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGSGGEIVLTQSPGTLSLSPG
ERATLSCRASQSVSQNHLAWYQQKPGQAPRLLIYLASRRHTGIPDRFSGSGSGTDFTLTISRLEPEDFAV
YYCQQSGRVPWTFGQGTKVEIKGGGGSGGGGSGGGGSAPTSSSTKKTQLQLEHLLLDLQMILNGINNY
KNPKLTRMLTFKFYMPKKATELKHLQCLEEELKPLEEVLNLAQSKNFHLRPRDLISNINVIVLELKGSETTF
MCEYADETATIVEFLNRWITFCQSIISTLT
SEQ ID NO: 22-Amino acid sequence of the flexible linker connecting the PEA2 diabody and human IL2
in the PEA2-IL2 conjugate, connecting p40 and p35 domains of the murine IL12, connecting p40 and p35
domains of the human IL12, connecting VH and VL of the anti-CD3 in the PEA2-anti-CD3 bispecific
antibody in a bispecific T cell engager format
GGGGSGGGGSGGGGS
SEQ ID NO: 23-Amino acid sequence of human IL2 (hIL2)
APTSSSTKKTQLQLEHLLLDLQMILNGINNYKNPKLTRMLTFKFYMPKKATELKHLQCLEEELKPLEEVLNL
AQSKNFHLRPRDLISNINVIVLELKGSETTFMCEYADETATIVEFLNRWITFCQSIISTLT
SEQ ID NO: 24-Amino acid sequence of the PEA2-mTNF conjugate
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGGGSGGGGSGGGGEIVLT
QSPGTLSLSPGERATLSCRASQSVSQNHLAWYQQKPGQAPRLLIYLASRRHTGIPDRFSGSGSGTDFTL
TISRLEPEDFAVYYCQQSGRVPWTFGQGTKVEIKSSSSGSSSSGSSSSGLRSSSQNSSDKPVAHVVANH
QVEEQLEWLSQRANALLANGMDLKDNQLVVPADGLYLVYSQVLFKGQGCPDYVLLTHTVSRFAISYQEK
VNLLSAVKSPCPKDTPEGAELKPWYEPIYLGGVFQLEKGDQLSAEVNLPKYLDFAESGQVYFGVIAL
SEQ ID NO: 25-Amino acid sequence of murine TNF (mTNF)
LRSSSQNSSDKPVAHVVANHQVEEQLEWLSQRANALLANGMDLKDNQLVVPADGLYLVYSQVLFKGQG
CPDYVLLTHTVSRFAISYQEKVNLLSAVKSPCPKDTPEGAELKPWYEPIYLGGVFQLEKGDQLSAEVNLP
KYLDFAESGQVYFGVIAL
SEQ ID NO: 26-Amino acid sequence of the PEA2-hIL12 conjugate
The linker sequences are underlined.
IWELKKDVYVVELDWYPDAPGEMVVLTCDTPEEDGITWTLDQSSEVLGSGKTLTIQVKEFGDAGQYTCH
KGGEVLSHSLLLLHKKEDGIWSTDILKDQKEPKNKTFLRCEAKNYSGRFTCWWLTTISTDLTFSVKSSRG
SSDPQGVTCGAATLSAERVRGDNKEYEYSVECQEDSACPAAEESLPIEVMVDAVHKLKYENYTSSFFIR
DIIKPDPPKNLQLKPLKNSRQVEVSWEYPDTWSTPHSYFSLTFCVQVQGKSKREKKDRVFTDKTSATVIC
RKNASISVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDELMQALNFNSETVPQKSSLEEP
DFYKTKIKLCILLHAFRIRAVTIDRVMSYLNASGSADGGSSAGGSDAGEVQLLESGGGLVQPGGSLRLSC
AASGFTFSRTAMSWVRQAPGKGLEWVSAIDYDGGVTYYADSVKGRFTISRDNSKNTLYLQMNSLRAED
TAVYYCAKLTYARFDYWGQGTLVTVSSGGSGGEIVLTQSPGTLSLSPGERATLSCRASQSVSQNHLAWY
QQKPGQAPRLLIYLASRRHTGIPDRFSGSGSGTDFTLTISRLEPEDFAVYYCQQSGRVPWTFGQGTKVEI
KSSSSGSSSSGSSSSGEVQLLESGGGLVQPGGSLRLSCAASGFTFSRTAMSWVRQAPGKGLEWVSAID
YDGGVTYYADSVKGRFTISRDNSKNTLYLQMNSLRAEDTAVYYCAKLTYARFDYWGQGTLVTVSSGGS
GGEIVLTQSPGTLSLSPGERATLSCRASQSVSQNHLAWYQQKPGQAPRLLIYLASRRHTGIPDRFSGSG
SGTDFTLTISRLEPEDFAVYYCQQSGRVPWTFGQGTKVEIK
SEQ ID NO: 27-Amino acid sequence of human IL12 (hIL 12)
The linker sequence is underlined
IWELKKDVYVVELDWYPDAPGEMVVLTCDTPEEDGITWTLDQSSEVLGSGKTLTIQVKEFGDAGQYTCH
KGGEVLSHSLLLLHKKEDGIWSTDILKDQKEPKNKTFLRCEAKNYSGRFTCWWLTTISTDLTFSVKSSRG
SSDPQGVTCGAATLSAERVRGDNKEYEYSVECQEDSACPAAEESLPIEVMVDAVHKLKYENYTSSFFIR
DIIKPDPPKNLQLKPLKNSRQVEVSWEYPDTWSTPHSYFSLTFCVQVQGKSKREKKDRVFTDKTSATVIC
RKNASISVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDELMQALNFNSETVPQKSSLEEP
DFYKTKIKLCILLHAFRIRAVTIDRVMSYLNAS
SEQ ID NO: 28-Amino acid sequence of the PEA2-hTNF conjugate
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGGGSGGGGSGGGGEIVLT
QSPGTLSLSPGERATLSCRASQSVSQNHLAWYQQKPGQAPRLLIYLASRRHTGIPDRFSGSGSGTDFTL
TISRLEPEDFAVYYCQQSGRVPWTFGQGTKVEIKSSSSGSSSSGSSSSGVRSSSRTPSDKPVAHVVANP
QAEGQLQWLNRRANALLANGVELRDNQLVVPSEGLYLIYSQVLFKGQGCPSTHVLLTHTISRIAVSYQTK
VNLLSAIKSPCQRETPEGAEAKPWYEPIYLGGVFQLEKGDRLSAEINRPDYLDFAESGQVYFGIIAL
SEQ ID NO: 29-Amino acid sequence of the soluble form of the extracellular domain of human TNFα
(hTNFα)
VRSSSRTPSDKPVAHVVANPQAEGQLQWLNRRANALLANGVELRDNQLVVPSEGLYLIYSQVLFKGQG
CPSTHVLLTHTISRIAVSYQTKVNLLSAIKSPCQRETPEGAEAKPWYEPIYLGGVFQLEKGDRLSAEINRPD
YLDFAESGQVYFGIIAL
SEQ ID NO: 30-Amino acid sequence of the IL2-PEA2-TNFα^{mut} conjugate
The linker sequences are underlined.
APTSSSTKKTQLQLEHLLLDLQMILNGINNYKNPKLTRMLTFKFYMPKKATELKHLQCLEEELKPLEEVLNL
AQSKNFHLRPRDLISNINVIVLELKGSETTFMCEYADETATIVEFLNRWITFCQSIISTLTGDGSSGGSGGA
SEVQLLESGGGLVQPGGSLRLSCAASGFTFSRTAMSWVRQAPGKGLEWVSAIDYDGGVTYYADSVKGR
FTISRDNSKNTLYLQMNSLRAEDTAVYYCAKLTYARFDYWGQGTLVTVSSGGGGSGGGGSGGGGEIVL
TQSPGTLSLSPGERATLSCRASQSVSQNHLAWYQQKPGQAPRLLIYLASRRHTGIPDRFSGSGSGTDFT
LTISRLEPEDFAVYYCQQSGRVPWTFGQGTKVEIKSSSSGSSSSGSSSSGVRSSSRTPSDKPVAHVVAN
PQAEGQLQWLNRAANALLANGVELRDNQLVVPSEGLYLIYSQVLFKGQGCPSTHVLLTHTISRIAVSYQT
KVNLLSAIKSPCQRETPEGAEAKPWYEPIYLGGVFQLEKGDRLSAEINRPDYLDFAESGQVYFGIIAL
SEQ ID NO: 31-Amino acid sequence of the linker between IL2 and PEA2 in the IL2-PEA2-TNFα^{mut}
conjugate
GDGSSGGSGGAS
SEQ ID NO 32-Amino acid sequence of the soluble form of the extracellular domain of human TNFα
(R32A) mutant
VRSSSRTPSDKPVAHVVANPQAEGQLQWLNRAANALLANGVELRDNQLVVPSEGLYLIYSQVLFKGQGC
PSTHVLLTHTISRIAVSYQTKVNLLSAIKSPCQRETPEGAEAKPWYEPIYLGGVFQLEKGDRLSAEINRPDY
LDFAESGQVYFGIIAL
SEQ ID NO: 33-Amino acid sequence of the PEA2-anti-CD3 (PUB4) bispecific antibody in bispecific T
cell engager format
The linker sequences are underlined.
EIVLTQSPGTLSLSPGERATLSCRASQSVSQNHLAWYQQKPGQAPRLLIYLASRRHTGIPDRFSGSGSGT
DFTLTISRLEPEDFAVYYCQQSGRVPWTFGQGTKVEIKGGGGSGGGGSGGGGSEVQLLESGGGLVQP
GGSLRLSCAASGFTFSRTAMSWVRQAPGKGLEWVSAIDYDGGVTYYADSVKGRFTISRDNSKNTLYLQ
MNSLRAEDTAVYYCAKLTYARFDYWGQGTLVTVSSGGGGSEVQLVESGGGLVQPGGSLRLSCAASGFT
FSTYAMNWVRQAPGKGLEWVGRIRSKYNNYATYYADSVKDRFTISRDDSKNSLYLQMNSLKTEDTAVYY
CVRHGNFGNSYVSWFAYWGQGTLVTVSSGGGGSGGGGSGGGGSQAVVTQEPSLTVSPGGTVTLTCR
SSTGAVTTSNYANWVQQKPGQAPRGLIGGTNKRAPWTPARFSGSLLGGKAALTITGAQAEDEADYYCAL
WYSNLWVFGGGTKLTVL
SEQ ID NO: 34-Amino acid sequence of the anti-PEA2-PUB4 bispecific antibody in scDb-scFv format
The linker sequences are underlined
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGSGGEIVLTQSPGTLSLSPG
ERATLSCRASQSVSQNHLAWYQQKPGQAPRLLIYLASRRHTGIPDRFSGSGSGTDFTLTISRLEPEDFAV
YYCQQSGRVPWTFGQGTKVEIKGGGGSGGGGSGGGGSEVQLLESGGGLVQPGGSLRLSCAASGFTF
SRTAMSWVRQAPGKGLEWVSAIDYDGGVTYYADSVKGRFTISRDNSKNTLYLQMNSLRAEDTAVYYCA
KLTYARFDYWGQGTLVTVSSGGSGGEIVLTQSPGTLSLSPGERATLSCRASQSVSQNHLAWYQQKPGQ
APRLLIYLASRRHTGIPDRFSGSGSGTDFTLTISRLEPEDFAVYYCQQSGRVPWTFGQGTKVEIKGSADG
GSSAGGSDAGEVQLVESGGGLVQPGGSLRLSCAASGFTFSTYAMNWVRQAPGKGLEWVGRIRSKYNN
YATYYADSVKDRFTISRDDSKNSLYLQMNSLKTEDTAVYYCVRHGNFGNSYVSWFAYWGQGTLVTVSS
GGGGSGGGGSGGGGSQAVVTQEPSLTVSPGGTVTLTCRSSTGAVTTSNYANWVQQKPGQAPRGLIGG
TNKRAPWTPARFSGSLLGGKAALTITGAQAEDEADYYCALWYSNLWVFGGGTKLTVL
SEQ ID NO: 35-Amino acid sequence of the linker between PEA2 and anti-CD3 in the PEA2-anti-CD3
bispecific antibody in the bispecific T cell engager format
GGGGS
SEQ ID NO: 36-Amino acid sequence connecting the hIL12 and the PEA2 scDb in the PEA2-hIL12
conjugate, and connecting PEA2 scDb and PUB4 scFv in the anti-CD3-PEA2 scDb-scFv bispecific
antibody
GSADGGSSAGGSDAG
SEQ ID NO: 37-C-terminal BirA target sequence
GLNDIFEAQKIEWHE
SEQ ID NO: 38-Amino acid sequence of the KSF heavy chain in IgG1 format
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKSPKVSLFDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 39-Amino acid sequence of the KSF light chain in IgG1 format
SSELTQDPAVSVALGQTVRITCQGDSLRSYYASWYQQKPGQAPVLVIYGKNNRPSGIPDRFSGSSSGNT
ASLTITGAQAEDEADYYCNSSPLNRLAVVFGGGTKLTVLGQPKAAPSVTLFPPSSEELQANKATLVCLISD
FYPGAVTVAWKADSSPVKAGVETTTPSKQSNNKYAASSYLSLTPEQWKSHKSYSCQVTHEGSTVEKTV
APTECS
SEQ ID NO: 40-Amino acid sequence of the PEA2 heavy chain in IgG4 format
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPCSRSTSES
TAALGCLVKDYFPEPVTVSWNSGALTSGVHTFPAVLQSSGLYSLSSVVTVPSSSLGTKTYTCNVDHKPS
NTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGK
SEQ ID NO: 41-Amino acid sequence of the PEA2 antibody in small immunoprotein format (SIP)
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGGGSGGGGSGGGGEIVLT
QSPGTLSLSPGERATLSCRASQSVSQNHLAWYQQKPGQAPRLLIYLASRRHTGIPDRFSGSGSGTDFTL
TISRLEPEDFAVYYCQQSGRVPWTFGQGTKVEIKSGGSGGPRAAPEVYAFATPEWPGSRDKRTLACLIQ
NFMPEDISVQWLHNEVQLPDARHSTTQPRKTKGSGFFVFSRLEVTRAEWEQKDEFICRAVHEAASPSQT
VQRAVSVNPESSRRGGC
SEQ ID NO: 42-Amino acid sequence of the G9 VH domain
The VH CDRs are underlined.
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKLTYARFDYWGQGTLVTVSS
SEQ ID NO: 43-Amino acid sequence of the G9 VL domain
The VL CDRs are underlined.
EIVLTQSPGTLSLSPGERATLSCRASQSVSSSYLAWYQQKPGQAPRLLIYGASSRATGIPDRFSGSGSGT
DFTLTISRLEPEDFAVYYCQQSGRVPWTFGQGTKVEIK
SEQ ID NO: 44-Amino acid sequence of the F7 VH domain
The VH CDRs are underlined.
EVQLLESGGGLVQPGGSLRLSCAASGFTFSRTAMSWVRQAPGKGLEWVSAISGSGGSTYYADSVKGRF
TISRDNSKNTLYLQMNSLRAEDTAVYYCAKLTYARFDYWGQGTLVTVSS
SEQ ID NO: 45-Amino acid sequence of the F7 VL domain
The VL CDRs are underlined.
EIVLTQSPGTLSLSPGERATLSCRASQSVSFPYLAWYQQKPGQAPRLLIYGASSRATGIPDRFSGSGSGT
DFTLTISRLEPEDFAVYYCQQSGRVPWTFGQGTKVEIK
SEQ ID NO: 46-Amino acid sequence of the KSF VH domain
The VH CDRs are underlined.
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKSPKVSLFDYWGQGTLVTVSS
SEQ ID NO: 47-Amino acid sequence of the KSF VL domain
The VL CDRs are underlined.
SSELTQDPAVSVALGQTVRITCQGDSLRSYYASWYQQKPGQAPVLVIYGKNNRPSGIPDRFSGSSSGNT
ASLTITGAQAEDEADYYCNSSPLNRLAVVFGGGTKLTVLG
SEQ ID NO: 48-Amino acid sequence of the “knob”-heavy chain in the PEA2-PUB4 IgG4-scFv (2 + 1)
bispecific antibody
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPCSRSTSES
TAALGCLVKDYFPEPVTVSWNSGALTSGVHTFPAVLQSSGLYSLSSVVTVPSSSLGTKTYTCNVDHKPS
NTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQV
YTLPPCQEEMTKNQVSLWCLVKGFYPSDIAVEWESNGQPENNYKTTPPVLDSDGSFFLYSRLTVDKSR
WQEGNVFSCSVMHEALHNHYTQKSLSLSLGKGSEVQLVESGGGLVQPGGSLRLSCAASGFTFSTYAMN
WVRQAPGKGLEWVGRIRSKYNNYATYYADSVKDRFTISRDDSKNSLYLQMNSLKTEDTAVYYCVRHGN
FGNSYVSWFAYWGQGTLVTVSSGGGGSGGGGSGGGGSQAVVTQEPSLTVSPGGTVTLTCRSSTGAV
TTSNYANWVQQKPGQAPRGLIGGTNKRAPWTPARFSGSLLGGKAALTITGAQAEDEADYYCALWYSNL
WVFGGGTKLTVL
SEQ ID NO: 49-Amino acid sequence of the “hole”-heavy chain in the PEA2-PUB4 IgG4-scFv (2 + 1)
bispecific antibody
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPCSRSTSES
TAALGCLVKDYFPEPVTVSWNSGALTSGVHTFPAVLQSSGLYSLSSVVTVPSSSLGTKTYTCNVDHKPS
NTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQV
CTLPPSQEEMTKNQVSLSCAVKGFYPSDIAVEWESNGQPENNYKTTPPVLDSDGSFFLVSRLTVDKSRW
QEGNVFSCSVMHEALHNHYTQKSLSLSLGK
SEQ ID NO: 50-Amino acid sequence of the heavy chain in the PEA2-PUB4 IgG4-(scFv)₂ (2 + 2)
bispecific antibody
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPCSRSTSES
TAALGCLVKDYFPEPVTVSWNSGALTSGVHTFPAVLQSSGLYSLSSVVTVPSSSLGTKTYTCNVDHKPS
NTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGGSEVQLVESGGGLVQPGGSLRLSCAASGFTFSTYAMNWV
RQAPGKGLEWVGRIRSKYNNYATYYADSVKDRFTISRDDSKNSLYLQMNSLKTEDTAVYYCVRHGNFGN
SYVSWFAYWGQGTLVTVSSGGGGSGGGGSGGGGSQAVVTQEPSLTVSPGGTVTLTCRSSTGAVTTSN
YANWVQQKPGQAPRGLIGGTNKRAPWTPARFSGSLLGGKAALTITGAQAEDEADYYCALWYSNLWVFG
GGTKLTVL
SEQ ID NO: 51-Amino acid sequence of the PUB4 VH domain
EVQLVESGGGLVQPGGSLRLSCAASGFTFSTYAMNWVRQAPGKGLEWVGRIRSKYNNYATYYADSVKD
RFTISRDDSKNSLYLQMNSLKTEDTAVYYCVRHGNFGNSYVSWFAYWGQGTLVTVSS
SEQ ID NO: 52-Amino acid sequence of the PUB4 VL domain
QAVVTQEPSLTVSPGGTVTLTCRSSTGAVTTSNYANWVQQKPGQAPRGLIGGTNKRAPWTPARFSGSL
LGGKAALTITGAQAEDEADYYCALWYSNLWVFGGGTKLTVL
SEQ ID NO: 53-Amino acid sequence of the PUB4 antibody in scFv format
The linker sequence is underlined.
EVQLVESGGGLVQPGGSLRLSCAASGFTFSTYAMNWVRQAPGKGLEWVGRIRSKYNNYATYYADSVKD
RFTISRDDSKNSLYLQMNSLKTEDTAVYYCVRHGNFGNSYVSWFAYWGQGTLVTVSSGGGGSGGGGS
GGGGSQAVVTQEPSLTVSPGGTVTLTCRSSTGAVTTSNYANWVQQKPGQAPRGLIGGTNKRAPWTPA
RFSGSLLGGKAALTITGAQAEDEADYYCALWYSNLWVFGGGTKLTVL
SEQ ID NO: 54-Amino acid sequence of the scDb_{PEA2} Fc Knob chain in scDb_{PEA2}-SCFv_{PUB4} with Fc KIH
bispecific antibody
The linker sequences are underlined.
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSGGSGGEIVLTQSPGTLSLSPG
ERATLSCRASQSVSQNHLAWYQQKPGQAPRLLIYLASRRHTGIPDRFSGSGSGTDFTLTISRLEPEDFAV
YYCQQSGRVPWTFGQGTKVEIKGGGGSGGGGSGGGGSEVQLLESGGGLVQPGGSLRLSCAASGFTF
SRTAMSWVRQAPGKGLEWVSAIDYDGGVTYYADSVKGRFTISRDNSKNTLYLQMNSLRAEDTAVYYCA
KLTYARFDYWGQGTLVTVSSGGSGGEIVLTQSPGTLSLSPGERATLSCRASQSVSQNHLAWYQQKPGQ
APRLLIYLASRRHTGIPDRFSGSGSGTDFTLTISRLEPEDFAVYYCQQSGRVPWTFGQGTKVEIKGSESK
YGPPCPPCPAPEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVYTLPPCQEEMT
KNQVSLWCLVKGFYPSDIAVEWESNGQPENNYKTTPPVLDSDGSFFLYSRLTVDKSRWQEGNVFSCSV
MHEALHNHYTQKSLSLSLGK
SEQ ID NO: 55-Amino acid sequence of the scFv_{PUB4} FC Hole chain in scDb_{PEA2}-SCFV_{PUB4} with Fc KIH
bispecific antibody
The linker sequence is underlined.
EVQLVESGGGLVQPGGSLRLSCAASGFTFSTYAMNWVRQAPGKGLEWVGRIRSKYNNYATYYADSVKD
RFTISRDDSKNSLYLQMNSLKTEDTAVYYCVRHGNFGNSYVSWFAYWGQGTLVTVSSGGGGSGGGGS
GGGGSQAVVTQEPSLTVSPGGTVTLTCRSSTGAVTTSNYANWVQQKPGQAPRGLIGGTNKRAPWTPA
RFSGSLLGGKAALTITGAQAEDEADYYCALWYSNLWVFGGGTKLTVLGSESKYGPPCPPCPAPEFLGGP
SVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVCTLPPSQEEMTKNQVSLSCAVKGFYPSDI
AVEWESNGQPENNYKTTPPVLDSDGSFFLVSRLTVDKSRWQEGNVFSCSVMHEALHNHYTQKSLSLSL
GK
SEQ ID NO: 56-Amino acid sequence of the scFv_{Pub4}-IgG_{4PEA2} Knob heavy chain in the scFv_{Pub4}-IgG_{4PEA2}
KIH bispecific antibody
The linker sequences are underlined.
EVQLVESGGGLVQPGGSLRLSCAASGFTFSTYAMNWVRQAPGKGLEWVGRIRSKYNNYATYYADSVKD
RFTISRDDSKNSLYLQMNSLKTEDTAVYYCVRHGNFGNSYVSWFAYWGQGTLVTVSSGGGGSGGGGS
GGGGSQAVVTQEPSLTVSPGGTVTLTCRSSTGAVTTSNYANWVQQKPGQAPRGLIGGTNKRAPWTPA
RFSGSLLGGKAALTITGAQAEDEADYYCALWYSNLWVFGGGTKLTVLGGGGSGGGGSGGGGSEVQLL
ESGGGLVQPGGSLRLSCAASGFTFSRTAMSWVRQAPGKGLEWVSAIDYDGGVTYYADSVKGRFTISRD
NSKNTLYLQMNSLRAEDTAVYYCAKLTYARFDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQVYTLPP
CQEEMTKNQVSLWCLVKGFYPSDIAVEWESNGQPENNYKTTPPVLDSDGSFFLYSRLTVDKSRWQEGN
VFSCSVMHEALHNHYTQKSLSLSLGK
SEQ ID NO: 57-Amino acid sequence of IgG_{4PEA2} “Hole” heavy chain in the scFv_{Pub4}-IgG_{4PEA2} KIH
bispecific antibody
EVQLLESGGGLVQPGGSLRLSCAASGFTFSRTAMSWVRQAPGKGLEWVSAIDYDGGVTYYADSVKGRF
TISRDNSKNTLYLQMNSLRAEDTAVYYCAKLTYARFDYWGQGTLVTVSSASTKGPSVFPLAPCSRSTSES
TAALGCLVKDYFPEPVTVSWNSGALTSGVHTFPAVLQSSGLYSLSSVVTVPSSSLGTKTYTCNVDHKPS
NTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQV
CTLPPSQEEMTKNQVSLSCAVKGFYPSDIAVEWESNGQPENNYKTTPPVLDSDGSFFLVSRLTVDKSRW
QEGNVFSCSVMHEALHNHYTQKSLSLSLGK
SEQ ID NO: 58-Amino acid sequence of the IgG_{4PEA2} light chain in the scFv_{Pub4}-IgG_{4PEA2} KIH
bispecific antibody
EIVLTQSPGTLSLSPGERATLSCRASQSVSQNHLAWYQQKPGQAPRLLIYLASRRHTGIPDRFSGSGSGT
DFTLTISRLEPEDFAVYYCQQSGRVPWTFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSF
NRGEC
SEQ ID NO: 59-Amino acid sequence of the heavy chain in the IgG_{4AE2P}-(SCFV_{PEA2})₂ bispecific antibody
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRYIAFDYWGQGTLVTVSSASTKGPSVFPLAPCSRSTSEST
AALGCLVKDYFPEPVTVSWNSGALTSGVHTFPAVLQSSGLYSLSSVVTVPSSSLGTKTYTCNVDHKPSN
TKVDKRVESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSRLTVDKSRWQ
EGNVFSCSVMHEALHNHYTQKSLSLSLGK
SEQ ID NO: 60-Amino acid sequence of the light chain in the IgG_{4AE2P}-(SCFV_{PEA2})₂ bispecific antibody
The linker sequences are underlined.
DIQMTQSPSSLSASVGDRVTITCRASQSISSYLNWYQQKPGKAPKLLIYAASSLQSGVPSRFSGSGSGTD
FTLTISSLQPEDFATYYCQQGGMPPDTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGECGGGGSGGGGSGGGGSEIVLTQSPGTLSLSPGERATLSCRASQSVSQNHLAWYQQKPGQAPRLL
IYLASRRHTGIPDRFSGSGSGTDFTLTISRLEPEDFAVYYCQQSGRVPWTFGQGTKVEIKGGGGSGGGG
SGGGGSEVQLLESGGGLVQPGGSLRLSCAASGFTFSRTAMSWVRQAPGKGLEWVSAIDYDGGVTYYA
DSVKGRFTISRDNSKNTLYLQMNSLRAEDTAVYYCAKLTYARFDYWGQGTLVTVSS
SEQ ID NO: 61-Amino acid sequence of the heavy chain in the IgG_{4AE2P}-(SCF_{VSm3E})₂ bispecific antibody
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRYIAFDYWGQGTLVTVSSASTKGPSVFPLAPCSRSTSEST
AALGCLVKDYFPEPVTVSWNSGALTSGVHTFPAVLQSSGLYSLSSVVTVPSSSLGTKTYTCNVDHKPSN
TKVDKRVESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSRLTVDKSRWQ
EGNVFSCSVMHEALHNHYTQKSLSLSLGK
SEQ ID NO: 62-Amino acid sequence of the light chain in the IgG_{4AE2P}-(SCF_{VSm3E})₂ bispecific antibody
The linker sequences are underlined.
DIQMTQSPSSLSASVGDRVTITCRASQSISSYLNWYQQKPGKAPKLLIYAASSLQSGVPSRFSGSGSGTD
FTLTISSLQPEDFATYYCQQGGMPPDTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFN
RGECGGGGSGGGGSGGGGSQVKLEQSGAEVVKPGASVKLSCKASGFNIKDSYMHWLRQGPGQRLEW
IGWIDPENGDTEYAPKFQGKATFTTDTSANTAYLGLSSLRPEDTAVYYCNEGTPTGPYYFDYWGQGTLV
TVSSGGGGSGGGGSGGGGENVLTQSPSSMSVSVGDRVTIACSASSSVPYMHWLQQKPGKSPKLLIYLT
SNLASGVPSRFSGSGSGTDYSLTITSVQPEDAATYYCQQRSSYPLTFGGGTKLEIK

### REFERENCES

A number of publications are cited above in order to more fully describe and disclose the invention and the state of the art to which the invention pertains. Full citations for these references are provided below. The entirety of each of these references is incorporated herein.
Batista et al., (1996), J. Exp. Med., 184:2197-205
Bird et al. (1988) Science, 242, 423-426
Blumental et al., Cancer Res (2005), 65, 8809
Chester et al., Lancet (1994), 343, 455
Fairhead et al., Methods Mol Biol. 2015; 1266:171-84
Garambois et al., BMC Cancer (2004), 4, 75
Habdel Nabi et al., J Nucl Med (1990), 31, 1975
Hammarstrom et al., Cancer Res (1989), 49, 4852
Hekman et al. J Nucl Med (2017) 58, 706
Holliger et al. (1993a), Proc. Natl. Acad. Sci. USA 90 6444-6448
Holliger and Winter, 1997, Cancer Immunol. Immunother. 45 (3-4), 128-30
Holt et al. (2003) Trends in Biotechnology 21, 484-490
Hu et al. (1996), Cancer Res., 56 (13): 3055-61
Huston et al. (1988) PNAS USA, 85, 5879-5883
Kabat et al., “Sequences of Proteins of Immunological Interest,” National Institutes of Health, Bethesda, Md. (1991)
Kontermann, mAbs, (2012) 4:2, 182-197.
Konterman and Muller, 1999, FEBS Letters 454:90-91.
Li et al., (1997), Protein Engineering, 10:731-736
Liu et al., (2022) MABs Vol. 14, NO. 1, e2073632 (13 pages)
McCafferty et al., (1990) Nature, 348, 552-554
Neumaier et al., Cancer Res (1990), 50, 2128
Pavoni et al., BMC Cancer (2006) 6,41
Pedersen et al., J Mol Biol (1994) 235, 959
Perez et al., Biotechnol Appl Biochem (2006), 43, 39
Qin et al., J Label Compd Radiopharm (2022) 65, 63
Reiter et al. (1996), Nature Biotech, 14, 1239-1245
Ridgway et al. (1996), Protein Eng, 9 (7): 617-21
Rijpkema et al., J Nucl Med (2014), 55, 1519
Sato et al., J Nucl Med (1999), 40, 685
Stein and Goldenberg, Mol Cancer Ther (2004), 12, 1559
Viti et al., Methods Enzymol, (2000), 326, 480
Wagener et al., J Immunol (1983), 130, 2302
Ward et al. (1989) Nature 341, 544-546
Yazaki et al., PEDS (2004), 17, 481

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A antibody molecule that binds the N-terminal domain of carcinoembryonic antigen (CEA), wherein the antibody molecule comprises a VH domain comprising a set of complementarity determining regions HCDR1, HCDR2 and HCDR3, and a VL domain comprising a set of complementarity determining regions LCDR1, LCDR2 and LCDR3, wherein:
the HCDR1, HCDR2 and HCDR3 comprise the amino acid sequences set forth in SEQ ID NOs 2, 3 and 4, respectively, and
the LCDR1, LCDR2 and LCDR3 comprise the amino acid sequences set forth in SEQ ID NOs 5, 6 and 7, respectively.

2. The antibody molecule according to claim 1, wherein the VH domain comprises the amino acid sequence set forth in SEQ ID NO: 8 and/or the VL domain comprises the amino acid sequence set forth in SEQ ID NO: 9.

3. The antibody molecule according to claim 1, wherein the antibody molecule comprises or consists of: a single chain Fv (scFv), a small immunoprotein (SIP), a diabody (Db), a single-chain diabody (scDb), or an IgG molecule.

4. The antibody molecule according to claim 3, wherein:
the antibody is in scFv format and comprises the amino acid sequence set forth in SEQ ID NO: 10;
the antibody is in diabody (Db) format and comprises the amino acid sequence set forth in SEQ ID NO: 12;
the antibody is in single-chain diabody (scDb) format and comprises the amino acid sequence set forth in SEQ ID NO: 13;
the antibody is in small immunoprotein (SIP) format and comprises the amino acid sequence set forth in SEQ ID NO: 41;
the antibody molecule is in IgG1 format and comprises the heavy chain amino acid sequence set forth in SEQ ID NO: 16, and the light chain amino acid sequence set forth in SEQ ID NO: 17; or
the antibody molecule is in IgG4 format and comprises the heavy chain amino acid sequence set forth in SEQ ID NO: 40, and the light chain amino acid sequence set forth in SEQ ID NO: 17.

5. The antibody molecule according to claim 1 wherein the antibody molecule is conjugated to a pro-inflammatory agent, a biocidal molecule, a cytotoxic molecule, or a radioisotope.

6.-21. (canceled)

22. The antibody molecule according to claim 5, wherein (i) the pro-inflammatory agent, biocidal molecule, cytotoxic molecule, or radioisotope is conjugated to the N-terminus or C-terminus of the antibody molecule; and/or (ii) the pro-inflammatory agent is a cytokine.

23. The antibody molecule according to claim 22, wherein the pro-inflammatory agent is interleukin 2 (IL2), interleukin 12 (IL12), or tumor necrosis factor alpha (TNFα) or a variant thereof.

24. The antibody molecule according to claim 23, wherein
(i) the antibody molecule is a single-chain diabody and is conjugated at its N-terminus to interleukin 12 (IL12), and wherein the conjugate comprises the amino acid sequence set forth in SEQ ID NO: 26;
(ii) the antibody molecule is a diabody and is conjugated at its C-terminus to interleukin 2 (IL2), and wherein the conjugate comprises the amino acid sequence set forth in SEQ ID NO: 21; or
(iii) the antibody molecule is an scFv and is conjugated at its C-terminus to tumor necrosis factor alpha (TNFα) or a variant thereof, and wherein a) the conjugate has the sequence set forth in SEQ ID NO: 28, or b) the scFv is further conjugated at its N-terminus to IL2, and the conjugate has the sequence set forth in SEQ ID NO: 30.

25. The antibody molecule according to claim 1, wherein the antibody is a bispecific antibody molecule and further comprises a second antigen-binding site which binds a T cell antigen.

26. The antibody molecule according to claim 25, wherein the T cell antigen is CD3 or CD28.

27. The antibody molecule of claim 26, wherein the second antigen-binding site comprises the VH domain sequence set forth in SEQ ID NO: 51 and/or the VL domain sequence set forth in SEQ ID NO: 52.

28. The antibody molecule according to claim 27, wherein the second antigen-binding site is an scFv and has the amino acid sequence set forth in SEQ ID NO: 53.

29. The antibody molecule according to claim 25, wherein:
the antibody molecule is in bispecific T-cell engager format and has the amino acid sequence set forth in SEQ ID NO: 33;
the antibody molecule is in scDb-scFv format and has the amino acid sequence set forth in SEQ ID NO: 34;
the antibody molecule is in IgG-scFv format and has the heavy chain amino acid sequences set forth in SEQ ID NOs: 48 and 49, and the light chain amino acid sequence set forth in SEQ ID NO: 17;
the antibody molecule is in IgG-(scFv)₂ format and has the heavy chain amino acid sequence set forth in SEQ ID NO: 50, and the light chain amino acid sequence set forth in SEQ ID NO: 17;
the antibody molecule is in scFv-IgG KIH format and has the heavy chain amino acid sequences set forth in SEQ ID NOs: 56 and 57, and the light chain amino acid sequence set forth in SEQ ID NO: 58;
the antibody molecule is in scDb-scFv with Fc KIH format and has the heavy chain amino acid sequence set forth in SEQ ID NO: 54 and the heavy chain amino acid sequence set forth in SEQ ID NO: 55; or
the antibody molecule is in IgG-(scFv)₂ format and has the heavy chain amino acid sequence set forth in SEQ ID NO: 59 and the light chain amino acid sequence set forth in SEQ ID NO: 60.

30. A method of treating cancer in a patient, comprising administration of a therapeutically effective amount of the antibody molecule of claim 1 to the patient.

31. The method of claim 30, further comprising:
(i) administering an anti-PD-1 antibody to the patient; or
(ii) administering a bispecific antibody molecule to the patient, wherein said bispecific antibody molecule binds CEA and a T cell antigen, wherein the T cell antigen is CD28.

32. The method of claim 31 (ii), wherein said bispecific antibody molecule has the heavy chain sequence of SEQ ID NO: 59 and the light chain sequence of SEQ ID NO: 60, or wherein said bispecific antibody molecule has the heavy chain sequence of SEQ ID NO: 61 and the light chain sequence of SEQ ID NO: 62.

33. A nucleic acid molecule or expression vector encoding the antibody molecule according to claim 1.

34. A host cell comprising the nucleic acid or vector of claim 33.

35. A method of producing an antibody molecule according to claim 1, comprising culturing a host cell comprising a nucleic acid expressing said antibody under conditions for expression of the antibody molecule and isolating and/or purifying the antibody molecule so expressed.

36. The antibody molecule according to claim 3, wherein the antibody is an IgG molecule selected from an IgG1 or IgG4 molecule.


SEQUENCE LISTING (62 sequences)

SEQ ID NO: 1 - protein, 116 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSIS

SEQ ID NO: 2 - protein, 10 aa, synthetic construct
GFTFSRTAMS

SEQ ID NO: 3 - protein, 17 aa, synthetic construct
AIDYDGGVTYYADSVKG

SEQ ID NO: 4 - protein, 8 aa, synthetic construct
LTYARFDY

SEQ ID NO: 5 - protein, 12 aa, synthetic construct
RASQSVSQNHLA

SEQ ID NO: 6 - protein, 7 aa, synthetic construct
LASRRHT

SEQ ID NO: 7 - protein, 9 aa, synthetic construct
QQSGRVPWT

SEQ ID NO: 8 - protein, 117 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSS

SEQ ID NO: 9 - protein, 108 aa, synthetic construct
EIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 10 - protein, 239 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 11 - protein, 14 aa, synthetic construct
GGGGSGGGGSGGGG

SEQ ID NO: 12 - protein, 230 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 13 - protein, 475 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 14 - protein, 5 aa, synthetic construct
GGSGG

SEQ ID NO: 15 - protein, 15 aa, synthetic construct
SSSSGSSSSGSSSSG

SEQ ID NO: 16 - protein, 447 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 215 aa, synthetic construct
EIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 1011 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMRPLKNSQVEVSWEYPDSWSTPHSYFSLKFFVRIQRKKEKMKETEEGCNQKGAFLVERTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSADIGGGAGGGGAGGGAEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 19 - protein, 15 aa, synthetic construct
DIGGGAGGGGAGGGA

SEQ ID NO: 20 - protein, 521 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMRPLKNSQVEVSWEYPDSWSTPHSYFSLKFFVRIQRKKEKMKETEEGCNQKGAFLVERTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 21 - protein, 378 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 15 aa, synthetic construct
GGGGSGGGGSGGGGS

SEQ ID NO: 23 - protein, 133 aa, Homo sapiens [sequence omitted]

SEQ ID NO: 24 - protein, 410 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 25 - protein, 156 aa, Mus musculus
LRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 26 - protein, 1008 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSADGGSSAGGSDAGEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIK

SEQ ID NO: 27 - protein, 518 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 411 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 29 - protein, 157 aa, Homo sapiens [sequence omitted]

SEQ ID NO: 30 - protein, 556 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGDGSSGGSGGASEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSSSSGSSSSGSSSSGVRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 31 - protein, 12 aa, synthetic construct
GDGSSGGSGGAS

SEQ ID NO: 32 - protein, 157 aa, Homo sapiens
VRSSSRTPSDKPVAHVVANPQAEGQLQWLNRAANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 33 - protein, 494 aa, synthetic construct
EIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGGGGSGGGGSGGGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 34 - protein, 739 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGGGGSGGGGSGGGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGSADGGSSAGGSDAGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 35 - protein, 5 aa, synthetic construct
GGGGS

SEQ ID NO: 36 - protein, 15 aa, synthetic construct
GSADGGSSAGGSDAG

SEQ ID NO: 37 - protein, 15 aa, synthetic construct
GLNDIFEAQKIEWHE

SEQ ID NO: 38 - protein, 448 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 40 - protein, 444 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 360 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGGGSGGGGSGGGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 42 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKLTYARFDYWGQGTLVTVSS

SEQ ID NO: 43 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQSGRVPWTFGQGTKVEIK

SEQ ID NO: 44 - protein, 117 aa, synthetic construct
EVQLLESGGGLVQPGGSLRLSCAASGFTFSRTAMSWVRQAPGKGLEWVSAISGSGGSTYYADSVKGRFTISRDNSKNTLYLQMNSLRAEDTAVYYCAKLTYARFDYWGQGTLVTVSS

SEQ ID NO: 45 - protein, 108 aa, synthetic construct
EIVLTQSPGTLSLSPGERATLSCRASQSVSFPYLAWYQQKPGQAPRLLIYGASSRATGIPDRFSGSGSGTDFTLTISRLEPEDFAVYYCQQSGRVPWTFGQGTKVEIK

SEQ ID NO: 46 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSS

SEQ ID NO: 47 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLG

SEQ ID NO: 48 - protein, 695 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 49 - protein, 444 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 50 - protein, 694 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 51 - protein, 125 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 52 - protein, 109 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 53 - protein, 249 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 54 - protein, 706 aa, synthetic construct
EVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGGGGSGGGGSGGGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSGGSGGEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 480 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 56 - protein, 708 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGSGGGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 444 aa, synthetic construct; identical to SEQ ID NO: 49

SEQ ID NO: 58 - protein, 215 aa, synthetic construct; identical to SEQ ID NO: 17

SEQ ID NO: 59 - protein, 443 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRYIAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 469 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEIVLTQSPGTLSLSPGERATLSCRASQSVSQNHLAWYQQKPGQAPRLLIYLASRRHTGIPDRFSGSGSGTDFTLTISRLEPEDFAVYYCQQSGRVPWTFGQGTKVEIKGGGGSGGGGSGGGGSEVQLLESGGGLVQPGGSLRLSCAASGFTFSRTAMSWVRQAPGKGLEWVSAIDYDGGVTYYADSVKGRFTISRDNSKNTLYLQMNSLRAEDTAVYYCAKLTYARFDYWGQGTLVTVSS

SEQ ID NO: 61 - protein, 443 aa, synthetic construct; identical to SEQ ID NO: 59

SEQ ID NO: 62 - protein, 469 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGGMPPDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGENVLTQSPSSMSVSVGDRVTIACSASSSVPYMHWLQQKPGKSPKLLIYLTSNLASGVPSRFSGSGSGTDYSLTITSVQPEDAATYYCQQRSSYPLTFGGGTKLEIK